SEQUENCE LISTING (77 sequences)

SEQ ID NO: 1 - DNA, 20 bp, Artificial Sequence
  Primer targeted to a variety of fungi
tgaggtgctacagttattac

SEQ ID NO: 2 - DNA, 21 bp, Artificial Sequence
  Primer targeted to a variety of fungi
gaggtgctacagttattacta

SEQ ID NO: 3 - DNA, 20 bp, Artificial Sequence
  Primer targeted to a variety of fungi
ggtatagmtcttaawatagc

SEQ ID NO: 4 - DNA, 20 bp, Artificial Sequence
  Primer targeted to a variety of fungi
aaaatagcatagaaaggtaa

SEQ ID NO: 5 - DNA, 425 bp, Aspergillus fumigatus
tgaggtgctacagttattactaatcttatgagtgctataccttgaataggtcaagatatagttgaatttatatgaggtggtttctctgtaaataatgctacattaaatagattctttgcattacatttcttattaccttttgttttagctgcattagtaataatgcatttaatagcaatgcacgatactgtaggatctggtaatcctttaggtatttctggtaattatgatagattacctttcgctccttactttgtatttaaagatttagttactgtatttattttctttatagtattatctgtatttgtattcttcatgcctaacgcattaggtgatagtgaaaattatgttatggctaatccaatgcaaactccacctgctattgttccggaatgatacttattacctttctatgctatttt

SEQ ID NO: 6 - DNA, 425 bp, Aspergillus flavus
tgaggtgctacagttattactaaccttatgagtgctataccttgaataggtcaagayatagttgaatttatttgaggaggtttctctgtaaataatgcaacattaaatagattctttgcattacatttcttattaccttttgtattagctgcattagctttaatgcatttaatygctatgcacgatactgtaggatctggtaatcctttaggtatatctggtaattatgatagattaccttttgctccatatttcatatttaaagatttagtwactatctttattttctttatagtattatctatatttgttttcttyatgcctaatgctttaggagatagtgaaaattatgttatggctaatccaatgcaaactccacctgctatygttccagaatgatacttattacctttctatgctatttt

SEQ ID NO: 7 - DNA, 425 bp, Aspergillus niger
tgaggtgctacagttattactaaccttatgagtgctataccatgaataggtcaagatatagttgagttcatatgaggwggtttctctgtaaataatgcaacattaaacagattctttgcattacacttcttattaccttttgtattagctgcattagctttaatgcacttaatagctatgcacgatacagtaggatcaggtaatccattaggaatatcaggtaattacgatagattaccttttgcaccatattttatatttaaagatttagtaactatctttattttctttattgtattatcaatatttgttttctttatgcctaatgcattaggagatagtgaaaattatgttatggctaayccaatgcaaactccacctgcwattgtaccagagtgatatcttttacctttctatgctatttt

SEQ ID NO: 8 - DNA, 426 bp, Aspergillus nidulans
tgaggtgctacagttattactaacctaatgagtgctataccttgaataggtcaagatattgttgagtttatttgaggaggtttytctgtaaataatgcaactttaaacagattctttgcattacatttcttattaccttttgtattagctgctttagcattaatgcatttaatagctatgcacgatacagtaggatcaggkaatcctttaggtatttcagctaattacgatagattaccttttgctccttattttatatttaaagatttaataactatatttatattctttattgtattatcaatatttgttttctttatgcctaatgctttaggtgatagtgaaaattatgttatggctaatcctatgcaaactccacctgctatagttccagaatgatatcttttacctttctatgctatttta

SEQ ID NO: 9 - DNA, 426 bp, Aspergillus terreus
tgaggtgctacagttattactaacttaatgagtgctataccttgaattggtcaagatatagttgaatttatttgaggaggtttctctgtaaataatgcaactttaaatagattctttgcattacatttcttattaccttttgtattagctgcwttagttattatgcacttaatagcaatgcacgatactgtaggatcaggtaatcctttaggtatatcaggtaactacgatagattacctttcgctccatatttcgtattcaaagatttagtaactatctttattttctttatagtattatctatatttgttttcttcatgcctaacgcattaggagacagtgaaaattatgttatggcaaacccaatgcaaacaccacctgctattgtaccagaatgatacttattaccattctatgctatttta

SEQ ID NO: 10 - DNA, 21 bp, Artificial Sequence
  Primer targeted to Aspergillus fumigatus
aggcatgaagaatacaaatac

SEQ ID NO: 11 - DNA, 22 bp, Artificial Sequence
  Primer targeted to Aspergillus flavus
tcctacagtatcgtgcatagcg

SEQ ID NO: 12 - DNA, 21 bp, Artificial Sequence
  Primer targeted to Aspergillus flavus
cctacagtatcgtgcatagca

SEQ ID NO: 13 - DNA, 24 bp, Artificial Sequence
  Primer targeted to Aspergillus niger
gctaatacaaaaggtaataagaag

SEQ ID NO: 14 - DNA, 25 bp, Artificial Sequence
  Primer targeted to Aspergillus nidulans
gcaaaaggtaatctatcgtaattag

SEQ ID NO: 15 - DNA, 24 bp, Artificial Sequence
  Primer targeted to Aspergillus terreus
cattctggtacaatagcaggtggt

SEQ ID NO: 16 - DNA, 437 bp, Aspergillus fumigatus IFM40804
tgaggtgctacagttattactaatcttatgagtgctataccttgaataggtcaagatatagttgaatttatatgaggtggtttctctgtaaataatgctacattaaatagattctttgcattacatttcttattaccttttgttttagctgcattagtaataatgcatttaatagcaatgcacgatactgtaggatctggtaatcctttaggtatttctggtaattatgatagattacctttcgctccttactttgtatttaaagatttagttactgtatttattttctttatagtattatctgtatttgtattcttcatgcctaacgcattaggtgatagtgaaaattatgttatggctaatccaatgcaaactccacctgctattgttccggaatgatacttattacctttctatgctattttaagatctatacc

SEQ ID NO: 17 - DNA, 425 bp, Aspergillus fumigatus IMF40806; identical to SEQ ID NO: 5

SEQ ID NO: 18 - DNA, 425 bp, Aspergillus fumigatus IMF40807; identical to SEQ ID NO: 5

SEQ ID NO: 19 - DNA, 437 bp, Aspergillus fumigatus IFM40819; identical to SEQ ID NO: 16

SEQ ID NO: 20 - DNA, 425 bp, Aspergillus fumigatus IFM41206; identical to SEQ ID NO: 5

SEQ ID NO: 21 - DNA, 425 bp, Aspergillus fumigatus IMF41392; identical to SEQ ID NO: 5

SEQ ID NO: 22 - DNA, 425 bp, Aspergillus fumigatus IFM45916; identical to SEQ ID NO: 5

SEQ ID NO: 23 - DNA, 425 bp, Aspergillus fumigatus IFM45917; identical to SEQ ID NO: 5

SEQ ID NO: 24 - DNA, 425 bp, Aspergillus fumigatus IFM46980; identical to SEQ ID NO: 5

SEQ ID NO: 25 - DNA, 425 bp, Aspergillus fumigatus IFM5355; identical to SEQ ID NO: 5

SEQ ID NO: 26 - DNA, 425 bp, Aspergillus flavus IFM40603
tgaggtgctacagttattactaaccttatgagtgctataccttgaataggtcaagacatagttgaatttatttgaggaggtttctctgtaaataatgcaacattaaatagattctttgcattacatttcttattaccttttgtattagctgcattagctttaatgcatttaatcgctatgcacgatactgtaggatctggtaatcctttaggtatatctggtaattatgatagattaccttttgctccatatttcatatttaaagatttagttactatctttattttctttatagtattatctatatttgttttctttatgcctaatgctttaggagatagtgaaaattatgttatggctaatccaatgcaaactccacctgctatcgttccagaatgatacttattacctttctatgctatttt

SEQ ID NO: 27 - DNA, 425 bp, Aspergillus flavus IFM40800
tgaggtgctacagttattactaaccttatgagtgctataccttgaataggtcaagatatagttgaatttatttgaggaggtttctctgtaaataatgcaacattaaatagattctttgcattacatttcttattaccttttgtattagctgcattagctttaatgcatttaatcgctatgcacgatactgtaggatctggtaatcctttaggtatatctggtaattatgatagattaccttttgctccatatttcatatttaaagatttagtaactatctttattttctttatagtattatctatatttgttttctttatgcctaatgctttaggagatagtgaaaattatgttatggctaatccaatgcaaactccacctgctattgttccagaatgatacttattacctttctatgctatttt

SEQ ID NO: 28 - DNA, 425 bp, Aspergillus flavus IFM41087; identical to SEQ ID NO: 26

SEQ ID NO: 29 - DNA, 437 bp, Aspergillus flavus IFM41933
tgaggtgctacagttattactaaccttatgagtgctataccttgaataggtcaagatatagttgaatttatttgaggaggtttctctgtaaataatgcaacattaaatagattctttgcattacatttcttattaccttttgtattagctgcattagctttaatgcatttaattgctatgcacgatactgtaggatctggtaatcctttaggtatatctggtaattatgatagattaccttttgctccatatttcatatttaaagatttagttactatctttattttctttatagtattatctatatttgttttcttcatgcctaatgctttaggagatagtgaaaattatgttatggctaatccaatgcaaactccacctgctatcgttccagaatgatacttattacctttctatgctattttaagatctatacc

SEQ ID NO: 30 - DNA, 425 bp, Aspergillus flavus IFM45909; identical to SEQ ID NO: 26

SEQ ID NO: 31 - DNA, 437 bp, Aspergillus flavus IFM45910
tgaggtgctacagttattactaaccttatgagtgctataccttgaataggtcaagacatagttgaatttatttgaggaggtttctctgtaaataatgcaacattaaatagattctttgcattacatttcttattaccttttgtattagctgcattagctttaatgcatttaatcgctatgcacgatactgtaggatctggtaatcctttaggtatatctggtaattatgatagattaccttttgctccatatttcatatttaaagatttagttactatctttattttctttatagtattatctatatttgttttctttatgcctaatgctttaggagatagtgaaaattatgttatggctaatccaatgcaaactccacctgctatcgttccagaatgatacttattacctttctatgctattttaagatctatacc

SEQ ID NO: 32 - DNA, 425 bp, Aspergillus flavus IFM45911; identical to SEQ ID NO: 26

SEQ ID NO: 33 - DNA, 425 bp, Aspergillus flavus IFM46870; identical to SEQ ID NO: 26

SEQ ID NO: 34 - DNA, 425 bp, Aspergillus flavus IFM5364; identical to SEQ ID NO: 26

SEQ ID NO: 35 - DNA, 437 bp, Aspergillus flavus IFM5366
tgaggtgctacagttattactaaccttatgagtgctataccttgaataggtcaagatatagttgaatttatttgaggaggtttctctgtaaataatgcaacattaaatagattctttgcattacatttcttattaccttttgtattagctgcattagctttaatgcatttaatcgctatgcacgatactgtaggatctggtaatcctttaggtatatctggtaattatgatagattaccttttgctccatatttcatatttaaagatttagtaactatctttattttctttatagtattatctatatttgttttctttatgcctaatgctttaggagatagtgaaaattatgttatggctaatccaatgcaaactccacctgctattgttccagaatgatacttattacctttctatgctattttaagatctatacc

SEQ ID NO: 36 - DNA, 425 bp, Aspergillus niger IFM40606
tgaggtgctacagttattactaaccttatgagtgctataccatgaataggtcaagatatagttgagttcatatgaggtggtttctctgtaaataatgcaacattaaacagattctttgcattacacttcttattaccttttgtattagctgcattagctttaatgcacttaatagctatgcacgatacagtaggatcaggtaatccattaggaatatcaggtaattacgatagattaccttttgcaccatattttatatttaaagatttagtaactatctttattttctttattgtattatcaatatttgttttctttatgcctaatgcattaggagatagtgaaaattatgttatggctaacccaatgcaaactccacctgctattgtaccagagtgatatcttttacctttctatgctatttt

SEQ ID NO: 37 - DNA, 425 bp, Aspergillus niger IFM41398
tgaggtgctacagttattactaaccttatgagtgctataccatgaataggtcaagatatagttgagttcatatgaggaggtttctctgtaaataatgcaacattaaacagattctttgcattacacttcttattaccttttgtattagctgcattagctttaatgcacttaatagctatgcacgatacagtaggatcaggtaatccattaggaatatcaggtaattacgatagattaccttttgcaccatattttatatttaaagatttagtaactatctttattttctttattgtattatcaatatttgttttctttatgcctaatgcattaggagatagtgaaaattatgttatggctaatccaatgcaaactccacctgcaattgtaccagagtgatatcttttacctttctatgctatttt

SEQ ID NO: 38 - DNA, 437 bp, Aspergillus niger IFM41399
tgaggtgctacagttattactaaccttatgagtgctataccatgaataggtcaagatatagttgagttcatatgaggaggtttctctgtaaataatgcaacattaaacagattctttgcattacacttcttattaccttttgtattagctgcattagctttaatgcacttaatagctatgcacgatacagtaggatcaggtaatccattaggaatatcaggtaattacgatagattaccttttgcaccatattttatatttaaagatttagtaactatctttattttctttattgtattatcaatatttgttttctttatgcctaatgcattaggagatagtgaaaattatgttatggctaatccaatgcaaactccacctgcaattgtaccagagtgatatcttttacctttctatgctattttaagatctatacc

SEQ ID NO: 39 - DNA, 425 bp, Aspergillus niger IFM46897; identical to SEQ ID NO: 37

SEQ ID NO: 40 - DNA, 425 bp, Aspergillus niger IFM5367; identical to SEQ ID NO: 37

SEQ ID NO: 41 - DNA, 425 bp, Aspergillus niger IFM5368; identical to SEQ ID NO: 37

SEQ ID NO: 42 - DNA, 425 bp, Aspergillus nidulans IFM41094
tgaggtgctacagttattactaacctaatgagtgctataccttgaataggtcaagatattgttgagtttatttgaggaggtttttctgtaaataatgcaactttaaacagattctttgcattacatttcttattaccttttgtattagctgctttagcattaatgcatttaatagctatgcacgatacagtaggatcaggtaatcctttaggtatttcagctaattacgatagattaccttttgctccttattttatatttaaagatttaataactatatttatattctttattgtattatcaatatttgttttctttatgcctaatgctttaggtgatagtgaaaattatgttatggctaatcctatgcaaactccacctgctatagttccagaatgatatcttttacctttctatgctatttt

SEQ ID NO: 43 - DNA, 425 bp, Aspergillus nidulans IFM46999; identical to SEQ ID NO: 42

SEQ ID NO: 44 - DNA, 437 bp, Aspergillus nidulans IFM47004
tgaggtgctacagttattactaacctaatgagtgctataccttgaataggtcaagatattgttgagtttatttgaggaggtttctctgtaaataatgcaactttaaacagattctttgcattacatttcttattaccttttgtattagctgctttagcattaatgcatttaatagctatgcacgatacagtaggatcagggaatcctttaggtatttcagctaattacgatagattaccttttgctccttattttatatttaaagatttaataactatatttatattctttattgtattatcaatatttgttttctttatgcctaatgctttaggtgatagtgaaaattatgttatggctaatcctatgcaaactccacctgctatagttccagaatgatatcttttacctttctatgctattttaagatctatacc

SEQ ID NO: 45 - DNA, 437 bp, Aspergillus nidulans IFM47006; identical to SEQ ID NO: 44

SEQ ID NO: 46 - DNA, 425 bp, Aspergillus nidulans IFM41395; identical to SEQ ID NO: 42

SEQ ID NO: 47 - DNA, 437 bp, Aspergillus terreus IMF40851
tgaggtgctacagttattactaacttaatgagtgctataccttgaattggtcaagatatagttgaatttatttgaggaggtttctctgtaaataatgcaactttaaatagattctttgcattacatttcttattaccttttgtattagctgcattagttattatgcacttaatagcaatgcacgatactgtaggatcaggtaatcctttaggtatatcaggtaactacgatagattacctttcgctccatatttcgtattcaaagatttagtaactatctttattttctttatagtattatctatatttgttttcttcatgcctaacgcattaggagacagtgaaaattatgttatggcaaacccaatgcaaacaccacctgctattgtaccagaatgatacttattaccattctatgctattktaagatctmtacc

SEQ ID NO: 48 - DNA, 437 bp, Aspergillus terreus IFM40852; identical to SEQ ID NO: 47

SEQ ID NO: 49 - DNA, 437 bp, Aspergillus terreus IFM41092
tgaggtgctacagttattactaacttaatgagtgctataccttgaattggtcaagatatagttgaatttatttgaggaggtttctctgtaaataatgcaactttaaatagattctttgcattacatttcttattaccttttgtattagctgctttagttattatgcacttaatagcaatgcacgatactgtaggatcaggtaatcctttaggtatatcaggtaactacgatagattacctttcgctccatatttcgtattcaaagatttagtaactatctttattttctttatagtattatctatatttgttttcttcatgcctaacgcattaggagacagtgaaaattatgttatggcaaacccaatgcaaacaccacctgctattgtaccagaatgatacttattaccattctatgctattktaagatctmtacc

SEQ ID NO: 50 - DNA, 437 bp, Aspergillus terreus IFM5372; identical to SEQ ID NO: 47

SEQ ID NO: 51 - DNA, 437 bp, Aspergillus terreus IFM40850; identical to SEQ ID NO: 47

SEQ ID NO: 52 - DNA, 434 bp, Candida kefyl IFM5773
tgaggtgctacagttattactaatttattctcagctattccattaattggacaagatattgtattatgattatgaggaggtttctcagtatctaaccctacaattcaaagattctttgcattccattacttagtaccatttattattgctgcttgtgtaattatgcatttaatggctttacatacacatggttcatcaaatcctttaggtgtaacaggtaatttagatagattaccaatgcatggatactttatttttaaagatttaattacagtatttgtattcttaattttcttctcattatttgtattcttctcacctaatacattaggacatcctgataactatattccaggtaatccattagtaacaccagcatcaattgtacctgaatgatacttattaccattttatgctatwttaagatctatacc

SEQ ID NO: 53 - DNA, 434 bp, Candida kefyl IFM5800
tgaggtgctacagttattactaatttattctcagctattccattaattggacaagatattgtattatgattatgaggaggtttctcagtatctaaccctacaattcaaagattctttgcattccattacttagtaccatttattattgctgcttgtgtaattatgcatttaatggctttacatacacatggttcatcaaatcctttaggtgtaacaggtaatttagatagattaccaatgcatggatactttatttttaaagatttaattacagtatttgtattcttaattttcttctcattatttgtattcttctcacctaatacattaggacatcctgataactatattccaggtaatccattagtaacaccagcatcaattgtacctgaatgatacttattaccattttatgctattttaagatctatacc

SEQ ID NO: 54 - DNA, 434 bp, Candida tropicalis IFM40018
tgaggtgctacagttattactaacttattatcagctataccatttataggtaatgatatagtaccatttatatgaggaggtttctctgtatcaaatcctacaatacaaagattttttgcattacattatttattaccatttattttagcagcattagtagtaatgcattttatagcattacatactcatggatcatctaatcctgtaggaatatcaagtaatttagatagaataccaatgcatagttactttatatttaaagatttaattactgtatttgtatttatattagtatttagtttatttgtattcttctcacctaatactttaggacatcctgataactatataccaggtaatcctatggttacacctgcatctatagtacctgaatgatatttattaccattctatgctatattaagatctatacc

SEQ ID NO: 55 - DNA, 434 bp, Candida tropicalis IFM46816
tgaggtgctacagttattacwaacttattatcagctataccatttataggtaatgatatagtaccatttatatgaggaggtttctctgtatcaaatcctacaatacaaagattttttgcattacattatttattaccatttattttagcagcattagtagtaatgcattttatagcattacatactcatggatcatctaatcctgtaggaatatcaagtaatttagatagaataccaatgcatagttactttatatttaaagatttaattactgtatttgtatttatattagtatttagtttatttgtattcttctcacctaatactttaggacatcctgataactatataccaggtaatcctatggttacacctgcatctatagtacctgaatgatatttattaccattctatgctatwttaagatctatacc

SEQ ID NO: 56 - DNA, 437 bp, Trichophyton rubrum IFM45593
tgaggtgctacagttattactaatttattaagtgctataccttgaattggtcaagatttagtagaatttatttgaggaggtttttctgtaaataatgctacattaaatagatttttttctttacattatttattaccttttatattagctgctttagttttaatgcatttaatagcattacatgatactgttggttctagtaatcctttaggtatttcaggtaattatgatagattaccttttgctccttactttttatttaaagatttagttactattttcttatttatgcttggtttatctatatttgttttctttatgcctaatgctttaggtgactgtgaaaattatgtaatggctaatcctatgcaaacaccagctgctattgttcctgaatgatatttattacctttttatgctatattaagatctatacc

SEQ ID NO: 57 - DNA, 22 bp, Artificial Sequence
  Primer targeted to a variety of fungi
tgrggwgcwacwgttattacta

SEQ ID NO: 58 - DNA, 23 bp, Artificial Sequence
  Primer targeted to a variety of fungi
ggwatagmwsktaawayagcata

SEQ ID NO: 59 - DNA, 20 bp, Artificial Sequence
  Primer targeted to a variety of fungi
ggtatagmwsktaawayagc

SEQ ID NO: 60 - DNA, 20 bp, Artificial Sequence
  Primer targeted to a variety of fungi
ggtatagmwgttaawatagc

SEQ ID NO: 61 - DNA, 20 bp, Artificial Sequence
  Primer targeted to a variety of fungi
ggtatagatcttaawatagc

SEQ ID NO: 62 - DNA, 21 bp, Artificial Sequence
  Primer targeted to Aspergillus terreus
ggtggtgtttgcattgggttt

SEQ ID NO: 63 - DNA, 25 bp, Artificial Sequence
  Primer targeted to Aspergillus nidulans
caataaagaatataaatatagttat

SEQ ID NO: 64 - DNA, 26 bp, Artificial Sequence
  Primer targeted to Aspergillus niger
tatcgtaattacctgatattcctaat

SEQ ID NO: 65 - DNA, 27 bp, Artificial Sequence
  Primer targeted to Aspergillus fumigatus
tgcgttaggcatgaagaatacaaatac

SEQ ID NO: 66 - DNA, 24 bp, Artificial Sequence
  Primer targeted to Aspergillus flavus
gcattagctttaatgcatttaatc

SEQ ID NO: 67 - DNA, 24 bp, Artificial Sequence
  Primer targeted to Aspergillus flavus
gcattagctttaatgcatttaatt

SEQ ID NO: 68 - DNA, 30 bp, Artificial Sequence
  Primer targeted to Aspergillus fumigatus
ctgtatttattttctttatagtattatctg

SEQ ID NO: 69 - DNA, 21 bp, Artificial Sequence
  Primer targeted to Aspergillus fumigatus
atagtattatctgtatttgta

SEQ ID NO: 70 - DNA, 26 bp, Artificial Sequence
  Primer targeted to Aspergillus niger
cattaaacagattctttgcattacac

SEQ ID NO: 71 - DNA, 21 bp, Artificial Sequence
  Primer targeted to Aspergillus niger
ggatcaggtaatccattagga

SEQ ID NO: 72 - DNA, 20 bp, Artificial Sequence
  Primer targeted to Aspergillus nidulans
aatcctttaggtatttcagc

SEQ ID NO: 73 - DNA, 28 bp, Artificial Sequence
  Primer targeted to Aspergillus nidulans
atttaaagatttaataactatatttata

SEQ ID NO: 74 - DNA, 24 bp, Artificial Sequence
  Primer targeted to Aspergillus terreus
gttatggcaaacccaatgcaaaca

SEQ ID NO: 75 - DNA, 21 bp, Artificial Sequence
  Primer targeted to Aspergillus terreus
atggcaaacccaatgcaaaca

SEQ ID NO: 76 - DNA, 434 bp, Cunninghamella bertholletiae
tggggagcaacagttattactaatttattatctgctattccttggattggtaaagatcttgtagaatttatttggggtggtttcagtgttgataatgctactttaaatcgattctttagtctacattatttattaccattcattcttgctgctttagcagttatgcatttaattgcaaaagaagaaaatggatcaagtaatccattaggaattacagctaatgctgatatgatttatatgcatccatattatatctttaaagatttagtaactattttcttattcttcttagtattaggattattcttattttatgctcctaataaattaggacatcctgataattatattccagctaatccaatgcaaactccagcttctattgttcctgagtggtatctattaccattttatgctattttaaswkctatwcc

SEQ ID NO: 77 - DNA, 434 bp, Mucor circinelloides
tggggagcaacagttattactaacctattatcggctatcccttggattggtaaagatttagtagaattcatctggggaggtttctctgtagataatgcgacattaaaccgattcttcagtcttcactacttattaccattcatcttagcagctttatctttaatgcatttaattgctaaagaagaaaatggatctaataatccactaggaattacagcaaacactgatatggtttatcttcatccttattacgtatttaaagatttagtaacaattttcttattcttcttagtattagctttattcttattctatgcacctaataaattaggtcatcctgataattatattccagctaacccaatgcaaactcctgcatctattgtaccagaatggtatctattacctttctatgctattttaaswkctatwcc

(19) United States
(12) Patent Application Publication
(10) Pub. No.: US 2002/0098487 A1
(43) Pub. Date: Jul. 25, 2002

(54) Materials and methods for detecting fungi

(75) Inventors: Koji Yokoyama, Chiba-shi (JP); Li Wang, Chiba-shi (JP)

Correspondence Address: BIRCH STEWART KOLASCH & BIRCH, PO BOX 747, FALLS CHURCH, VA, 22040-0747, US

(73) Assignee: SS Pharmaceutical Co., Ltd.

(21) Appl. No.: 09/809,323

(22) Filed: Mar. 16, 2001

Related U.S. Application Data
(62) Division of application No. 09/068,395, filed on May 8, 1998, now abandoned.
Application No. 09/068,395 is a 371 of international application No. PCT/JP97/03164, filed on Sep. 9, 1997.

(30) Foreign Application Priority Data
Sep. 9, 1996 (JP) 260310/96

(51) Int. Cl. C12Q 1/68; C07H 21/04

(52) U.S. Cl. 435/6; 536/23.7; 536/24.3

(57) ABSTRACT

Nucleic acids for detecting fungi, which have sequences shown in SEQ ID NOs. 3, 57 to 75 are disclosed. The nucleic acids for detecting fungi may be used as probes or primers for gene amplification. In case of using them as primers, detection and identification of fungi belonging to the genus Aspergillus may be attained because the sizes of the amplified nucleic acid fragments vary depending on the species of the fungi belonging to the genus Aspergillus if the primers are appropriately selected.

### DESCRIPTION

Materials and Methods for Detecting Fungi

1. Technical Field

The present invention relates to a nucleic acid for detecting fungi and a method for detecting fungi using the same.

2. Background Art

Major pathogenic fungi causing deep mycosis include those belonging to the genus Candida (hereinafter referred to as “C.” for short), to the genus Aspergillus (hereinafter referred to as “A.” for short) and to the genus “Cryptococcus” (hereinafter referred to as “Cr.” for short). Major pathogenic fungi belonging to the genus Candida include C. albicans, C. kefyr, C. glabrata and C. tropicalis, and major pathogenic fungi belonging to the genus Cryptococcus include Cr. neoformans. Other pathogenic fungi causing deep mycosis, which are as important as those belonging to the genus Candida, are those belonging to the genus Aspergillus. The species causing deep mycosis, which belong to the genus Aspergillus include A. fumigatus, A. flavus, A. niger and niger group, A. nidulans and A. terreus. It is desired to quickly detect, group and identify these species.

In recent years, deep mycosis in the form of opportunistic infectious disease increases among immunodeficient patients. Since immunodeficient patients often have serious underlying diseases, it is necessary to identify the etiologic fungus at an early stage and to perform a proper therapy. Diagnosis of deep mycosis is based on the blood culture method. However, this method has a drawback in that the detection sensitivity is not satisfactory. Although diagnostics employing antibodies have been commercially available, diagnosis employing antibodies are useless for immunodeficient patients. Under these circumstances, methods for directly detecting antigens and other fungal components were developed recently. For example, methods for diagnosis by detecting mannan, D-arabinitol, glucan or the like has been developed. On the other hand, with the progress of molecular biology, methods for diagnosis comprising extracting DNAs of various pathogenic fungi followed by detection of nucleic acids by using PCR method have been developed. In the field of deep mycosis, a method for diagnosis by detecting ribosomal RNA has been proposed (Clinical Pathology, Vol. 43, Supplemental Edition, p.119, 1995).

### DISCLOSURE OF THE INVENTION

An object of the present invention is to provide a nucleic acid used for detecting pathogenic fungi causing deep mycosis, especially the fungi belonging to the genus Aspergillus, and to provide a method for detecting pathogenic fungi causing deep mycosis, especially the fungi belonging to the genus Aspergillus, by using the nucleic acid according to the present invention, which method is simple and quick, and has high specificity.

The present inventors intensively studied the genes of the pathogenic fungi causing deep mycosis, especially the fungi belonging to the genus Aspergillus, and turned attention to the mitochondrial cytochrome b genes. The present inventors prepared various nucleic acids for amplifying parts of the mitochondrial cytochrome b genes. Among the nucleic acids, the present inventors searched primers by which the fungal cytochrome b gene can be amplified. As a result, using the nucleic acids having the nucleotide sequences shown in SEQ ID NOs. 1-4 in the SEQUENCE LISTING, the present inventors succeeded in amplifying parts of the mitochondrial cytochrome b genes of fungi and in determining the nucleotide sequences of the amplified regions. The present inventors further studied based on the determined nucleotide sequences, to discover sequences which are specific to each of the species of the fungi belonging to the genus Aspergillus. Based on the specific sequences, the present inventors designed primers each of which is specific to the respective species. For example, the present inventors provided primers specific to A. fumigatus, which have the nucleotide sequences shown in SEQ ID NO. 10, 65, 68 and 69 in the SEQUENCE LISTING; primers specific to A. flavus which have the nucleotide sequences shown in SEQ ID NO. 11, 12, 66 and 67; primer specific to A. niger, which have the nucleotide sequences shown in SEQ ID NO. 13, 64, 70 and 71; primers specific to A. nidulans, which have the nucleotide sequences shown in SEQ ID NO. 14, 63, 72 and 73; and primers specific to A. terreus, which have the nucleotide sequences shown in SEQ ID NO. 15, 62, 74 and 75.

Based on these, the present inventors further prepared nucleic acids for detecting, grouping or identifying the fungi belonging to the genus Aspergillus simply, quickly, specifically and with high sensitivity, and established a method for detection using the nucleic acids, thereby completing the present invention. The present invention further provides a primer having the nucleotide sequence shown in SEQ ID NO. 57, which is an improvement of the primers having the nucleotide sequences shown in SEQ ID Nos. 1 and 2. By this primer, Cunninghamella bertholletiae and Mucor circinelloides can be detected in addition to the above-mentioned fungi.

That is, the present invention provides a primer for amplifying a fragment of mitochondrial cytochrome b gene of fungi, consisting essentially of a nucleic acid fragment of which nucleotide sequence is a sequence of not less than 10 consecutive nucleotides in the nucleotide sequence shown in SEQ ID NO. 57 (provided that one or more thymine at optional site(s) may be substituted with uracil) or in the nucleotide sequence complementary thereto. The present invention also provides a primers for amplifying a fragment of mitochondrial cytochrome b gene of fungi, consisting essentially of a nucleic acid fragment of which nucleotide sequences are sequences of not less than 10 consecutive nucleotides in the nucleotide sequence shown in SEQ ID NO. 3 or 58-61 (provided that one or more thymine at optional site(s) may be substituted with uracil) or in the nucleotide sequence complementary thereto. The present invention also provides nucleic acids for detecting Aspergillus fumigatus (SEQ ID NO. 5), Aspergillus flavus (SEQ ID NO. 6), Aspergillus niger (SEQ ID NO. 7), Aspergillus nidulans (SEQ ID NO. 8) or Aspergillus terreus (SEQ ID NO. 9), each of which consists essentially of a nucleic acid fragment of which nucleotide sequence is 10 to 100 consecutive nucleotides in the nucleotide sequence shown in SEQ ID NO. 5, 6, 7, 8 or 9 (provided that one or more thymine at optional site(s) may be substituted with uracil) or in the nucleotide sequence complementary thereto. The present invention further provides nucleic acids for detection of Aspergillus fumigatus, each of which consists essentially of a nucleic acid fragment of which nucleotide sequence is a sequence of not less than 10 consecutive nucleotides in the nucleotide sequence shown in SEQ ID NO. 5, the 3′-end of said nucleic acid being the 24nt “T”, 99nt “T”, 144nt “T”, 252nt “C”, 277nt “G”, 304nt “G”, 312nt “A” or 393nt “G” in the nucleotide sequence shown in SEQ ID NO. 5, or each of which consists essentially of a nucleic acid fragment of which nucleotide sequence is a sequence of not less than 10 consecutive nucleotides in the nucleotide sequence complementary to the sequence shown in SEQ ID NO. 5, of which 3′-end is the base that pairs with any of said 3′-ends of said nucleic acids. The present invention further provides a nucleic acid for detection of Aspergillus flavus, which consists essentially of a nucleic acid fragment of which nucleotide sequence is a sequence of not less than 10 consecutive nucleotides in the nucleotide sequence shown in SEQ ID NO. 6, the 3′-end of the nucleic acid being the 174nt “Y” in the nucleotide sequence shown in SEQ ID NO. 6, or each of which consists essentially of a nucleic acid fragment of which nucleotide sequence is a sequence of not less than 10 consecutive nucleotides in the nucleotide sequence complementary to the sequence shown in SEQ ID NO. 6, of which 3′-end is the base that pairs with any of said 3′-ends of said nucleic acids. The present invention further provides nucleic acids for detection of Aspergillus niger, each of which consists essentially of a nucleic acid fragment of which nucleotide sequence is a sequence of not less than 10 consecutive nucleotides in the nucleotide sequence shown in SEQ ID NO. 7, the 3′-end of the nucleic acid being the 42nt “A”, 69nt “C”, 126nt “C”, 207nt “A”, 213nt “A”, 246nt “A” or 396nt “G” in the nucleotide sequence shown in SEQ ID NO. 7, or each of which consists essentially of a nucleic acid fragment of which nucleotide sequence is a sequence of not less than 10 consecutive nucleotides in the nucleotide sequence complementary to the sequence shown in SEQ ID NO. 7, of which 3′-end is the base that pairs with any of said 3′-ends of said nucleic acids. The present invention further provides nucleic acids for detection of Aspergillus nidulans, each of which consists essentially of a nucleic acid fragment of which nucleotide sequence is a sequence of not less than 10 consecutive nucleotides in the nucleotide sequence shown in SEQ ID NO. 8, the 3′-end of the nucleic acid being the 60nt “tT”, 221nt “C”, 271nt “A”, 285nt “A”, 366nt “T” or 387nt “A” in the nucleotide sequence shown in SEQ ID NO. 8, or each of which consists essentially of a nucleic acid fragment of which nucleotide sequence is a sequence of not less than 10 consecutive nucleotides in the nucleotide sequence complementary to the sequence shown in SEQ ID NO. 8, of which 3′-end is the base that pairs with any of said 3′-ends of said nucleic acids. The present invention further provides nucleic acids for detection of Aspergillus terreus, each of which consists essentially of a nucleic acid fragment of which nucleotide sequence is a sequence of not less than 10 consecutive nucleotides in the nucleotide sequence shown in SEQ ID NO. 9, the 3′-end of the nucleic acid being the 25nt “T”, 48nt “T”, 162nt “T”, 225nt “C”, 261nt “C”, 339nt “C”, 360nt “A”, 375nt “A” or 411nt “A” in the nucleotide sequence shown in SEQ ID NO. 9, or each of which consists essentially of a nucleic acid fragment of which nucleotide sequence is a sequence of not less than 10 consecutive nucleotides in the nucleotide sequence complementary to the sequence shown in SEQ ID NO. 9, of which 3′-end is the base that pairs with any of said 3′-ends of said nucleic acids. The present invention further provides a method for detecting and/or identifying fungi belonging to the genus Aspergillus comprising amplifying a fragment of mitochondrial cytochrome b gene of fungi belonging to the genus Aspergillus by a gene amplification method using at least one forward primer selected from the group consisting of nucleic acid fragments of which nucleotide sequences are sequences of not less than 10 consecutive nucleotides in the nucleotide sequences shown in SEQ ID NOs. 1, 2 and 57 (provided that one or more thymine at optional site(s) may be substituted with uracil), and at least one reverse primer selected from the group consisting of nucleic acid fragments of which nucleotide sequences are sequences of not less than 10 consecutive nucleotides in the nucleotide sequences complementary to the sequences shown in SEQ ID NOs. 11-15, and 62-65 (provided that one or more thymine at optional site(s) may be substituted with uracil). The present invention further provides a method for detecting and/or identifying fungi belonging to the genus Aspergillus comprising amplifying a fragment of mitochondrial cytochrome b gene of fungi belonging to the genus Aspergillus by a gene amplification method using at least one forward primer selected from the group consisting of nucleic acid fragments of which nucleotide sequences are sequences of not less than 10 consecutive nucleotides in the nucleotide sequences shown in SEQ ID NOs. 66 to 75 (provided that one or more thymine at optional site(s) may be substituted with uracil), and at least one reverse primer selected from the group consisting of nucleic acid fragments of which nucleotide sequences are sequences of not less than 10 consecutive nucleotides in the nucleotide sequences complementary to the sequences shown in SEQ ID NOs. 3, 4, and 58 to 61 (provided that one or more thymine at optional site(s) may be substituted with uracil). The present invention still further provides a method for detecting and/or identifying fungi belonging to the genus Aspergillus comprising amplifying a fragment of mitochondrial cytochrome b gene of fungi belonging to the genus Aspergillus by a gene amplification method using at least one forward primer selected from the group consisting of nucleic acid fragments of which nucleotide sequences are sequences of not less than 10 consecutive nucleotides in the nucleotide sequences shown in SEQ ID NOs. 1, 2 and 57 (provided that one or more thymine at optional site(s) may be substituted with uracil), and at least one reverse primer selected from the group consisting of nucleic acid fragments of which nucleotide sequences are sequences of not less than 10 consecutive nucleotides in the nucleotide sequences complementary to the sequences shown in SEQ ID NOs. 3, 4, and 58 to 61 (provided that one or more thymine at optional site(s) may be substituted with uracil).

By the present invention, detection, grouping and identification of the pathogenic fungi, especially those belonging to the genus Aspergillus, can be made quickly, simply, specifically and with high sensitivity, of which grouping and identification hitherto have been time-consuming. The nucleic acids according to the present invention may be used either as primers or as probes for detection. Further, the products amplified by using the primers may be detected with the probes or restriction enzymes, thereby the sensitivity and specificity are further promoted. Thus, the significance of the present invention in the clinical field is large.

### BRIEF DESCRIPTION OF THE DRAWING

FIG. 1 is a schematic view showing the electrophoresis pattern obtained by amplifying mitochondrial cytochrome b genes of various fungi by PCR using various primers which are examples according to the present invention, and electrophoresing the amplified product.

FIG. 2 is a schematic view showing the electrophoresis pattern obtained by amplifying mitochondrial cytochrome b genes of various fungi by PCR using various primers which are other examples according to the present invention, and electrophoresing the amplified product.

FIG. 3 is a schematic view showing the electrophoresis pattern obtained by amplifying mitochondrial cytochrome b genes of various fungi by PCR using various primers which are still other examples according to the present invention, and electrophoresing the amplified product.

FIG. 4 is a schematic view showing the electrophoresis pattern obtained by amplifying mitochondrial cytochrome b genes of various fungi by PCR using various primers which are still other examples according to the present invention, and electrophoresing the amplified product.

### BEST MODE FOR CARRYING OUT THE INVENTION

Mitochondrial cytochrome b gene fragment of fungi may be amplified by hybridizing and elongating the chain of a pair of nucleic acids, one of which being selected from the group consisting of the nucleic acids having the nucleotide sequences shown in SEQ ID NOs. 1, 2 and 57, and another one of which being selected from the group consisting of the nucleic acids complementary to the nucleic acids having the nucleotide sequences shown in SEQ ID NOs. 3, 4 and 58 to 61, with the mitochondrial cytochrome b gene of fungi such as those belonging to the genus Aspergillus or Candida; and repeating the cycle including the extension reaction of the primers. Among the above-mentioned sequences, those shown in SEQ ID NOs. 1, 2 and 4 were designed based on the sequence of the mitochondrial cytochrome b gene of A. nidulans (Waring, R. B. et al., Cell 27, 4-11 (1981)). On the other hand, there are a plurality of nucleic acids having the nucleotide sequence shown in SEQ ID NO. 57, 3 or 58-61 (the nucleotide sequences shown in SEQ ID NOs. 57, 3, 58-61 are represented by general formulae, respectively). Although each of the nucleic acids having the sequence shown in one of these SEQ ID NOs. may be used individually, it is preferred to use a mixture of the nucleic acids shown by each general formula. By using the mixture, it was attained for the first time to amplify the mitochondrial cytochrome b gene of fungi other than those belonging to Aspergillus (excluding A. terreus), for example, A. terreus, those belonging to the genus Candida, Trichophyton, Cunninghamella or Mucor. The nucleic acids consisting essentially of a part of the sequence shown in SEQ ID NO. 57, 3 or 58-61, which part is not less than 10 consecutive nucleotides in the respective sequences may be employed as primers. However, it is preferred to employ primers having the full length of the sequences shown in these SEQ ID Nos., respectively.

Using the above-described various types of nucleic acids, fragments of mitochondrial cytochrome b genes of a number of strains of various fungi were amplified and nucleotide sequences thereof were determined. The sequences each of which is consentaneous to each species are shown in SEQ ID NOs. 5-9. The sequences shown in SEQ ID NOs. 5, 6, 7, 8 and 9 are the sequences of the amplified DNA fragments originated from A. fumigatus, A. flavus, A. niger, A. nidulans and A. terreus, respectively. Using these amplified fragments as templates, and using primers specific to each fungal species, which primers are hereinbelow described, amplification of nucleic acids may be carried out. Alternatively, by digesting the amplified product with one or more restriction enzymes and comparing the pattern of the generated fragments with standard patterns, the product formed by extension of primers may be detected (PCR-RFLP).

The nucleic acids consisting essentially of the nucleotide sequences which are optional 10 to 100 consecutive nucleotides, preferably 15 to 30 consecutive nucleotides, in the nucleotide sequences mentioned above may be used as nucleic acids for detection of the above-mentioned fungal species, respectively. The nucleic acids for detection include primers for amplifying nucleic acids and probes. In cases where the nucleic acids are used as probes, those labeled by a well-known method are used.

As concretely described in the examples below, the present inventors amplified the genes of a number of strains of the fungi belonging to the genus Aspergillus, determined the sequences of the amplified genes and carefully compared the sequences, to discover sites each of which is specific to each species. Examples of the specific sites (1 base) include the 24nt “T”, 99nt “T”, 144nt “T”, 252nt “C”, 277nt “G”, 304nt “G”, −312nt “A” and the 393nt “G” in the sequence shown in SEQ ID NO. 5 for A. fumigatus; the 174nt “Y” in the sequence shown in SEQ ID NO. 6 for A. flavus; the 69nt “C”, the 126nt “C”, the 207nt “A”, 213nt “A”, 246nt “A” and 396nt “G” in the sequence shown in SEQ ID NO. 7 for A. niger; the 60nt “T”, 221nt “C”, 271nt “A”, 285nt “A”, 366nt “T” and 387nt “A” in the sequence shown in SEQ ID NO. 8 for A. nidulans; and the 25nt “T”, 48nt “T”, 162nt “T”, 225nt “C”, 261nt “C”, 339nt “C”, 360nt “A”, 375nt “A” and 411nt “A” in the sequence shown in SEQ ID NO. 9 for A. terreus. These specific sites also exist in the complementary sequences of the sequences shown in SEQ ID NOs. 5-9, the bases of these specific sites in the complementary sequences being complementary to the bases mentioned above. By using an oligonucleotide whose 3′-end is the above-mentioned specific site (1 base) as a primer for amplifying the nucleic acid, the mitochondrial cytochrome b gene fragment of the corresponding species alone is amplified. Therefore, each species may be specifically detected, in other words, identification of the species may be carried out.

Examples of the sequences of the nucleic acids (used as reverse primers) which may be employed as primers in combination with the nucleic acid having the sequence shown in SEQ ID NO. 1, 2 and/or 57 (used as forward primers) are shown in SEQ ID NOs. 10-15 and 62-65. The 3′-end of each of these nucleic acids is one of the specific sites mentioned above in the sequences of SEQ ID NOs. 5-9, and these nucleic acids have not less than 10 bases. That is, the nucleic acids having the sequence shown in SEQ ID NOs. 10 and 65, respectively (both of these sequences are complementary chains of SEQ ID NO. 5, of which 3′-ends are the “C” which pairs with the 304nt “G” of SEQ ID NO. 5), are for detecting Aspergillus fumigatus. The nucleic acids having the sequence shown in SEQ ID NOs. 11 and 12, respectively (both of these sequences are complementary chains of SEQ ID NO. 6, of which 3′-ends are the “G” and “A”, respectively, which pair with the 174nt “Y” (C or T) of SEQ ID NO. 6), are for detecting Aspergillus flavus. The nucleic acids having the sequence shown in SEQ ID NO. 13 (complementary chain of SEQ ID NO. 7, of which 3′-end is the “G” which pairs with the 126nt “C” of SEQ ID NO. 7) and SEQ ID NO. 64 (complementary chain of SEQ ID NO. 7, of which 3′-end is the “T” which pairs with the 207nt “A” of SEQ ID NO. 7) are for detecting Aspergillus niger. The nucleic acids having the sequence shown in SEQ ID NO. 14 (complementary chain of SEQ ID NO. 8, of which 3′-end is the “G” which pairs with the 221nt “C” of SEQ ID NO. 8) and SEQ ID NO. 63 (complementary chain of SEQ ID NO. 8, of which 3′-end is the “T” which pairs with the 271nt “A” of SEQ ID NO. 8) are for detecting Aspergillus nidulans. The nucleic acids having the sequence shown in SEQ ID NO. 15 (complementary chain of SEQ ID NO. 9, of which 3′-end is the “T” which pairs with the 375nt “A” of SEQ ID NO. 9) and SEQ ID NO. 62 (complementary chain of SEQ ID NO. 9, of which 3′-end is the “T” which pairs with the 360nt “A” of SEQ ID NO. 9) are for detecting Aspergillus terreus.

On the other hand, primers for amplifying nucleic acids, by which identification of species can be carried out, may be designed by combining nucleic acids having the sequences shown in SEQ ID NO. 3, 4, 58, 59, 60 and/or 61 (used as reverse primers) and one of the above-mentioned nucleic acids, whose 3′-end is one of the above-mentioned specific sites (1 base). For example, the nucleic acid for detecting A. fumigatus is selected from the nucleic acids whose 3′-ends are any of the 24nt “T”, 99nt “T”, 144nt “T”, 252nt “C”, 277nt “G”, 304nt “G”, 312nt “A” and 393nt “G” in the sequence shown in SEQ ID NO. 5, which have not less than 10 bases. The nucleic acid for detecting A. flavus is selected from the nucleic acids whose 3′-ends are the 174nt “Y” in the sequence shown in SEQ ID NO. 6, which have not less than 10 bases. The nucleic acid for detecting A. niger is selected from the nucleic acids whose 3′-ends are any of the 42nt A 69nt “C”, 126nt “C”, 207nt “A”, 213nt “A”, 246nt “A” and 396nt “G” in the sequence shown in SEQ ID NO. 7, which have not less than 10 bases. The nucleic acid for detecting A. nidulans is selected from the nucleic acids whose 3′-ends are any of the 60nt “T”, 221nt “C”, 271nt “A”, 285nt “A”, 366nt “T” and 387nt “A” in the sequence shown in SEQ ID NO. 8, which have not less than 10 bases. The nucleic acid for detecting A. terreus is selected from the nucleic acids whose 3′-ends are any of the 25nt “T”, 48nt “T”, 162nt “T”, 225nt “C”, 261nt “C”, 339nt “C”, 360nt “A”, 375nt “A” and 411nt “A” in the sequence shown in SEQ ID NO. 9, which have not less than 10 bases. Preferred examples of these primers which are used as forward primers are shown in SEQ ID NOs. 66-75. The nucleic acids having the sequences shown in SEQ ID NOs. 66 and 67 (both of which 3′-ends are the 174nt “Y” (C or T) of SEQ ID NO. 6) are for detecting Aspergillus flavus. The nucleic acids having the sequences shown in SEQ ID NO. 68 (of which 3′-end is the 304nt “G” of SEQ ID NO. 5) and SEQ ID NO. 69 (of which 3′-end is the 312nt “A” of SEQ ID NO. 5) are for detecting Aspergillus fumigatus. The nucleic acids having the sequences shown in SEQ ID NO. 70 (of which 3′-end is the 126nt “C” of SEQ ID NO. 7) and SEQ ID NO. 71 (of which 3′-end is the 213nt “A” of SEQ ID NO. 7) are for detecting Aspergillus niger. The nucleic acids having the sequences shown in SEQ ID NOs. 72 (of which 3′-end is the 221nt “C” of SEQ ID NO. 8) and SEQ ID NO. 73 (of which 3′-end is the 285nt “A” of SEQ ID NO. 8) are for detecting Aspergillus nidulans. The nucleic acids having the sequences shown in SEQ ID NOs. 74 and 75 (both of which 3′-ends are the 375nt “A” of SEQ ID NO. 9) are for detecting Aspergillus fumigatus.

These oligonucleotides may be used not only as primers, but also as probes specific to each species after attaching an appropriate label thereto. The above-mentioned nucleic acids may be used as primers or probes as long as they have consecutive not less than 10 bases starting from the above-mentioned 3′-ends. The number of bases of the nucleotides is preferably 10 to 100, more preferably 15 to 30. As for the nucleic acids having the parts of the sequences shown in SEQ ID NOs. 10-15 and 62-75, it is preferred that they have the full length of these sequences.

In the nucleotide sequences of the nucleic acids according to the present invention, which are shown in the SEQUENCE LISTING, thymine (T) may be uracil (U). That is, the nucleic acids according to the present invention include both DNAs and RNAs. Further, the nucleic acid according to the present invention may be a DNA which contains uridine residue “U” in place of “T” at optional site(s), and may be an RNA which contains thymine residue “T” in place of “U” at optional site(s). Further, complementary chains of the nucleic acids according to the present invention are also within the scope of the present invention. This is because that the DNAs of fungi are double-stranded, so that the complementary chain of the nucleic acid according to the present invention can also hybridize with the complementary chain of the nucleic acid with which the nucleic acid according to the present invention hybridizes. Still further, as long as the nucleic acid can be applied to the above-described uses, the nucleic acids having one or more point mutations such as deletion, insertion and substitution, or those having modified nucleotide(s) are also within the scope of the present invention.

The method for obtaining the nucleic acids according to the present invention is not restricted. Preferably, they are chemically synthesized. By this method, the nucleic acids with uniform quality may be obtained easily and inexpensively in large scale. Such chemical synthesis may be carried out by a conventional method such as the phosphite method (phosphoamidite method) or the phosphotriester method (phosphoric acid triester method).

The method for detecting, grouping or identifying a fungus belonging to the genus Aspergillus according to the present invention is characterized by using the above-described nucleic acid for detecting, grouping or identifying a fungus belonging to the genus Aspergillus. The method includes (1) the method in which the nucleic acid is used as a probe, so as to detect, group or identify a fungus belonging to the genus Aspergillus, and (2) the method in which the nucleic acid is used as a primer for PCR or the like and the amplified product is measured, so as to detect, group or identify a fungus belonging to the genus Aspergillus. The nucleic acid used in these methods may be a labeled nucleic acid. Preferably, the nucleic acid is a labeled nucleic acid because the fungi belonging to the genus Aspergillus may be detected more easily. Examples of the label include antigens, haptens, enzymes, fluorescent substances, luminescent substances, enzyme substrates, radioactive substances and insoluble carriers. The label may be attached to either the end of the nucleic acid or to an inner portion of the sequence. The label may be attached to any of the saccharide, phosphate and base moieties.

The method for detecting, grouping or identifying fungi belonging to the genus Aspergillus using the nucleic acid according to the present invention as probes or primers include, for example, the following methods (1) and (2).

(1) In this method, the nucleic acid for detecting, grouping or identifying fungi belonging to the genus Aspergillus is used as a probe. That is, the nucleic acid is hybridized with the target nucleic acid in a sample and then the probe is detected, thereby detecting the target nucleic acid. In this method, it is preferred to use the nucleic acid for detecting, grouping or identifying fungi belonging to the genus Aspergillus, which is labeled with the above-mentioned label. This method may be carried out by hybridizing the labeled nucleic acid as a probe with the gene of a fungus belonging to the genus Aspergillus in a sample, and then detecting the hybrid between the probe and the gene of Aspergillus, or the non-hybridized probe by an appropriate method for detecting the label in the probe.

The hybridization between the gene of Aspergillus in the sample and the probe may be carried out by pretreating the sample to purify nucleic acids, mixing the resultant with the labeled nucleic acid as a probe, and incubating the mixture at a temperature between room temperature and 70° C. for 10 minutes to 48 hours. The target nucleic acid may be amplified by using the nucleic acid according to the present invention as a primer.

(2) In this method, using the nucleic acid for detecting fungi belonging to the genus Aspergillus according to the present invention as a primer, extension reaction of the primer is carried out using a DNA polymerase or the like, thereby specifically amplifying the cytochrome b gene fragment of a fungus belonging to the genus Aspergillus alone, and detecting the amplified product, thereby detecting, grouping or identifying Aspergillus. In this method, as the primer, the nucleic acid according to the present invention labeled with the above-mentioned label may also be used. An example of the gene amplification method is the PCR method. However, the gene amplification method is not restricted to the PCR method, but any gene amplification method in which a short oligonucleotide is used as a primer for initiation of the gene synthesis may be employed. The method for detecting Aspergillus according to the present invention may be carried out by detecting the amplified product, that is, the cytochrome b gene fragment. Alternatively, detection of Aspergillus may also be carried out by digesting the amplified cytochrome b gene fragment with one or more restriction enzymes and comparing the pattern of the sizes of the generated fragments with standard patterns. In this case, one restriction enzyme may be used or a plurality of restriction enzymes may be used in combination. The method for detecting the amplified cytochrome b gene fragment or the gene fragments generated by digesting the amplified product with one or more restriction enzymes is not restricted, and ordinary methods for detecting genes (e.g., electrophoresis) may be employed. The amplified product may be, for example, separated by electrophoresis, and the amplified gene may easily be detected as a band which is clear enough to be specifically detected. In cases where a plurality of primers are used in order to detecting, grouping or identifying a plurality of pathogenic fungi, the primers may be designed such that bands corresponding to different molecular weights are detected for the respective pathogenic fungi, thereby enabling the grouping or identification of each pathogenic fungus. By using deoxyribonucleotides (dATP, dTTP, dGTP, dCTP) or by using ribonucleotides (ATP, UTP, GTP, CTP) which are labeled with an appropriate label as the materials for the amplification reaction, the amplified product may be directly detected with high sensitivity. Alternatively, by using a labeled nucleic acid obtained by labeling the nucleic acid for detecting, grouping or identifying Aspergillus according to the present invention, the amplified product may be directly detected with high sensitivity. In these cases, preferred examples of the labels include radioactive substances, fluorescent substances, luminescent substances and luminescence-inducing substances.

An example of the method for detecting, grouping or identifying Aspergillus using the nucleic acid according to the present invention will now be described in more detail. Nucleic acids are extracted from the sample and nucleic acid-extension reaction such as PCR is carried out using the nucleic acid having the sequence shown in SEQ ID NO. 2 and a mixture of the nucleic acids having the sequences shown in SEQ ID NOs. 10-15 as primers. In this method, if A. fumigatus exists in the sample, a nucleic acid having a length of 323 bp is amplified; if A. flavus exists, nucleic acid having a length of 193 bp or 194 bp is amplified; if A. niger exists, a nucleic acid having a length of 148 bp is amplified; if A. nidulans exists, a nucleic acid having a length of 244 bp is amplified; and if A. terreus exists, a nucleic acid having a length of 397 bp is amplified, respectively. By separating and detecting these nucleic acids having different molecular weights by electrophoresis or the like, what species of Aspergillus exists in the sample can be determined. In cases where nucleic acid-extension reaction such as PCR is carried out using the nucleic acid having the sequence shown in SEQ ID NO. 2 and a mixture of the nucleic acids having the sequences shown in SEQ ID NOs. 62-65 as primers, if A. fumigatus exists in the sample, a nucleic acid having a length of 329 bp is amplified; if A. flavus exists, nucleic acid having a length of 193 bp or 194 bp is amplified; if A. niger exists, a nucleic acid having a length of 231 bp is amplified; if A. nidulans exists, a nucleic acid having a length of 294 bp is amplified; and if A. terreus exists, a nucleic acid having a length of 379 bp is amplified, respectively. In cases where nucleic acid-extension reaction such as PCR is carried out using the nucleic acid having the sequence shown in SEQ ID NO. 57 (mixture) and a mixture of the nucleic acids having the sequences shown in SEQ ID NOs. 62-65 as primers, if A. fumigatus exists in the sample, a nucleic acid having a length of 330 bp is amplified; if A. flavus exists, nucleic acid having a length of 194 bp or 195 bp is amplified; if A. niger exists, a nucleic acid having a length of 232 bp is amplified; if A. nidulans exists, a nucleic acid having a length of 295 bp is amplified; and if A. terreus exists, a nucleic acid having a length of 380 bp is amplified, respectively. In cases where nucleic acid-extension reaction such as PCR is carried out using the nucleic acids having the sequences shown in SEQ ID NO. 3, 58, 59, 60 and/or 61 (each sequence is a mixture) and a mixture of the nucleic acids having the sequences shown in SEQ ID NOs. 66-75 as primers, if A. fumigatus exists in the sample, nucleic acidS having lengths of 163 bp (by SEQ ID NO. 68) and 146 bp (by SEQ ID NO. 69) are amplified; if A. flavus exists, nucleic acid having a length of 287 bp is amplified; if A. niger exists, nucleic acids having lengths of 337 bp (by SEQ ID NO. 70) and 245 bp (by SEQ ID NO. 71) are amplified; if A. nidulans exists, nucleic acids having lengths of 236 bp (by SEQ ID NO. 72) and 180 bp (by SEQ ID NO. 73) are amplified; and if A. terreus exists, nucleic acids having lengths of 86 bp (by SEQ ID NO. 74) and 83 bp (by SEQ ID NO. 75) are amplified, respectively. The nucleic acids having sequences shown in SEQ ID NOs. 66-75 include two primers per each Aspergillus species. Therefore, by selecting one primer per each Aspergillus species, and using the selected totally 5 primers as a mixture, the above-mentioned 5 Aspergillus species may be simultaneously detected and identified.

The reagent kit for detecting Aspergillus, which utilizes the present invention, is characterized by including the nucleic acid for detecting Aspergillus according to the present invention or a labeled nucleic acid obtained by labeling the nucleic acid according to the present invention. As long as the kit includes the above-mentioned nucleotide, the kit is within the scope of the present invention, and the kit may further comprise other components such as a reagent for detecting a label, and a buffer solution. The nucleic acid in the reagent kit according to the present invention may be used as either a primer or a probe. The method in which the nucleic acid as a primer and the method in which the nucleic acid as a probe are different only in the means for detecting the target nucleic acid, and the essential features of these methods about detection of Aspergillus are the same. In cases where the nucleic acid is used as a probe, the reagent kit may be used for detection of Aspergillus by the above-described method (1), and in cases where the nucleic acid is used as a primer, the reagent kit may be used for detection of Aspergillus by the above-described method (2).

In cases where the nucleic acid in the reagent kit is used as a probe, the reagent kit according to the present invention may include a reagent for detecting the label and a buffer solution, in addition to the nucleic acid or labeled nucleic acid for detecting Aspergillus. In cases where the nucleic acid is used as a primer, the reagent kit according to the present invention may include a nucleic acid-synthesizing enzyme (e.g., DNA polymerase, RNA polymerase, reverse transcriptase and the like), deoxyribonucleotides (dATP, dTTP, dGTP and dCTP), ribonucleotides (ATP, UTP, GTP and CTP), one or more restriction enzymes, buffer solution and the like. The nucleic acid according to the present invention may be bound to a solid carrier and used as a capturing probe. In this case, using the capturing probe and a labeled probe in combination, sandwich assay may be carried out. Alternatively, the target nucleic acid may be labeled and captured. Further, the nucleic acid may be labeled with biotin, and after hybridization, the nucleic acid may be captured by an avidin-bound carrier. In the sandwich assay, if the nucleic acid according to the present invention is used as one of the probes, specific measurement may be obtained by virtue of the nucleic acid according to the present invention, so that there is no problem even if the specificity of the other nucleic acid is a little bit low.

The present invention will now be described by way of examples thereof. It should be noted that the present invention is not restricted to the examples described below.

### EXAMPLE 1

### Synthesis of Various DNAs

Synthesis of DNAs having the sequences shown in SEQ ID NOs. 1 to 4, 10 to 15 and 57 to 75 in the SEQUENCE LISTING were entrusted to a commercial DNA synthesis service. The DNAs having the sequences shown in SEQ ID NOs. 1 to 4, 10 to 15 and 57 to 75 will be referred to as Nucleic Acid 1 to 4, 10 to 15 and 57 to 75, respectively.

### EXAMPLE 2

### Amplification Reaction of Cytochrome b Genes of A. fumigatus

Cells of strains of A. fumigatus (A. fumigatus IFM 40804, 40806, 40807, 40819, 41206, 41392, 45916, 45917, 46980, 5355) were treated with 75% ethanol to kill the cells. After collecting the cells of each of the strains by centrifugation at 1500×g for 10 minutes, about 40 ml of extraction buffer (0.9 M sorbitol, 10 mM EDTA, 10 mM Tris-HCl buffer, pH7.1) was added to the cells of each strain and the resultant was well mixed. The mixture was then centrifuged at 1500×g for 10 minutes and the supernatant was discarded. Then 30 ml of the same buffer was added and the supernatant generated by the centrifugation as mentioned above was discarded, followed by addition of 9 ml of the same buffer. To the resultant, 1 ml of solution of Zymolyase (commercially available from SEIKAGAKU CORPORATION) (10 mg/ml) in the same buffer was added and the resulting mixture was incubated at 37° C. for 1 hour. Glass beads (0.9-1.3 mm) were added in the precipitation of cells, and the cells were disrupted by a vortex mixer for 1-2 minutes. The resultant was centrifuged at 1500×g for 10 minutes and the recovered supernatant was centrifuged at 20,000×g for 15 minutes to obtain a precipitate. The precipitate was washed with the extraction buffer and centrifuged again at 20,000×g for 15 minutes, to obtain mitochondrial fragment of each strain of A. fumigatus. To each of the thus obtained mitochondrial fragments, 0.5 ml of 1 mg/ml of protease K was added and the resultant was incubated at 37° C. for 1 hour. To this, 1 ml of a mixture of phenol/chloroform/isoamyl alcohol (25:24:1) was added and the mixture was well agitated by shaking, followed by centrifugation at 1500×g for 10 minutes to recover the supernatant. To the supernatant, 1 ml of phenol saturated with water was added and the mixture was well agitated by shaking, followed by centrifugation at 1500×g for 10 minutes to recover the supernatant. To the obtained supernatant, 0.1 ml of 3 M sodium acetate/0.1 M magnesium chloride solution was added and 3 ml of ethanol was added, followed by leaving the resultant to stand at −20° C. overnight. The resultant was centrifuged at 20,000×g for 10 minutes to recover a precipitate and this precipitate was washed with 75% ethanol at −20° C., followed by centrifugation at 1500×g for 10 minutes. The supernatant was discarded to obtain nucleic acids. The obtained nucleic acids were dried and stored at −20° C.

Using these DNAs as templates, and using the pair of DNA 1 (SEQ ID NO. 1) and DNA 3 (SEQ ID NO. 3; for IFM40804, 40819) or the pair of DNA 1 and DNA 4 (SEQ ID NO. 4; for IFM40806, 40807, 41206, 41392, 45916, 45917, 46980, 5355) as primers, PCR was performed under the conditions described below, to amplify a part of each cytochrome b gene. The PCR was performed using TaKaRa PCR Amplification Kit (R011) commercially available from TAKARA SHUZO CO., LTD. and a DNA amplifier (MIR-D30) commercially available from SANYO.

The composition of the reaction mixture was as follows:

| | | |
|---|---|---|
| ×10 PCR buffer | 5 | μl |
| (contained in kit) | | |
| PCR dNTP mixture solution | 4 | μl |
| (contained in kit) | | |
| Taq DNA polymerase | 1 | μl (2.5 U) |
| (contained in kit) | | |
| DNA 1 | 1 | μl |
| DNA 3 or 4 | 1 | μl |
| Mitochondrial DNA | 1 | μl |
| Water | balance to total volume of 50 | μl |

The reaction conditions were as follows:

Denaturing: 94° C., 1 minute

Annealing: 50° C., 1 minute

Extension: 72° C., 2 minutes

Each of the amplified DNAs was separated and purified by agarose gel electrophoresis and the nucleotide sequence thereof was determined by the following method.

That is, using a DNA sequencer (ABI Prism 377) commercially available from PERKIN ELMER, the sequence was determined by the dye terminator method according to the instructions by the manufacturer of the DNA sequencer. DNA 2 (SEQ ID NO. 2) was used as the forward primer for sequencing, and DNA 3 (for IFM40804, 40819) or DNA 4 (for IFM40806, 40807, 41206, 41392, 45916, 45917, 46980, 5355) was used as the reverse primer for sequencing. The determined nucleotide sequences are shown in SEQ ID NOs. 16 to 25 in the SEQUENCE LISTING.

### EXAMPLE 3

### Amplification Reaction of Cytochrome b Genes of A. flavus

By the same method as in Example 2, parts of the cytochrome b genes of strains of A. flavus (A. flavus IFM40603, 40800, 41087, 41933, 45909, 45910, 45911, 46870, 5364, 5366) were amplified by PCR and sequences thereof were determined. As the primer pair, the pair of DNA 2 and DNA 3 (for IFM41933, 45910, 5366) or the pair of DNA 2 and DNA 4 (for IFM40603, 40800, 41087, 45909, 45911, 46870, 5364) was used. In sequencing, DNA 2 was used as the forward primer and DNA 3 (for IFM41933, 45910, 5366) or DNA 4 (for IFM40603, 40800, 41087, 45909, 45911, 46870, 5364) was used as the reverse primer. The determined nucleotide sequences are shown in SEQ ID NOs. 26 to 35 in the SEQUENCE LISTING.

### EXAMPLE 4

### Amplification Reaction of Cytochrome b Genes of A. niger

By the same method as in Example 2, parts of the cytochrome b genes of strains of A. niger (A. niger IFM40606, 41398, 41399, 46897, 5367, 5368) were amplified by PCR and sequences thereof were determined. As the primer pair, the pair of DNA 2 and DNA 3 (for IFM41399) or the pair of DNA 2 and DNA 4 (for IFM40606, 41398, 46897, 5367, 5368) was used. In sequencing, DNA 2 was used as the forward primer and DNA 3 (for IFM41399) or DNA 4 (for IFM40606, 41398, 46897, 5367, 5368) was used as the reverse primer. The determined nucleotide sequences are shown in SEQ ID NOs. 36 to 41 in the SEQUENCE LISTING.

### EXAMPLE 5

### Amplification Reaction of Cytochrome b Genes of A. niger

By the same method as in Example 2, parts of the cytochrome b genes of strains of A. nidulans (A. nidulans IFM41094, 46999, 47004, 47006, 41395) were amplified by PCR and sequences thereof were determined. As the primer pair, the pair of DNA 2 and DNA 3 (for IFM47004, 47006) or the pair of DNA 2 and DNA 4 (for IFM41094, 46999, 41395) was used. In sequencing, DNA 2 was used as the forward primer and DNA 3 (for IFM47004, 47006) or DNA 4 (for IFM41094, 46999, 41395) was used as the reverse primer. The determined nucleotide sequences are shown in SEQ ID NOs. 42 to 46 in the SEQUENCE LISTING.

### EXAMPLE 6

### Amplification Reaction of Cytochrome b Genes of A. terreus

By the same method as in Example 2, parts of the cytochrome b genes of strains of A. terreus (A. terreus IFM40851, 40852, 41092, 5372, 40850) were amplified by PCR and sequences thereof were determined. As the primer pair, the pair of DNA 1 and DNA 3 was used. In sequencing, DNA 2 was used as the forward primer and DNA 3 was used as the reverse primer. The determined nucleotide sequences are shown in SEQ ID NOs. 47 to 51 in the SEQUENCE LISTING.

### EXAMPLE 7

### Amplification Reaction of Cytochrome b Genes of C. kefyl

By the same method as in Example 2, parts of the cytochrome b genes of strains of C. kefyl (C. kefyl IFM5733, 5800) were amplified by PCR and sequences thereof were determined. As the primer pair, the pair of DNA 2 and DNA 3 was used. In sequencing, DNA 2 was used as the forward primer and DNA 3 was used as the reverse primer. The determined nucleotide sequences are shown in SEQ ID NOs. 52 and 53 in the SEQUENCE LISTING.

### EXAMPLE 8

### Amplification Reaction of Cytochrome b Genes of C. tropicalis

By the same method as in Example 2, parts of the cytochrome b genes of strains of C. tropicalis (C. tropicalis IFM40018, 46816) were amplified by PCR and sequences thereof were determined. As the primer pair, the pair of DNA 1 and DNA 3 prepared in Example 1 was used. In sequencing, DNA 2 was used as the forward primer and DNA 3 was used as the reverse primer. The determined nucleotide sequences are shown in SEQ ID NOs. 54 and 55 in the SEQUENCE LISTING.

### EXAMPLE 9

### Amplification Reaction of Cytochrome b Gene of Trichophyton rubrum

By the same method as in Example 2, a part of the cytochrome b gene of a strain of Trichophyton rubrum (T. rubrum IFM45593) was amplified by PCR and sequence thereof was determined. As the primer pair, the pair of DNA 1 and DNA 3 prepared in Example 1 was used. In sequencing, DNA 2 was used as the forward primer and DNA 3 was used as the reverse primer. The determined nucleotide sequence is shown in SEQ ID NO. 56 in the SEQUENCE LISTING.

### EXAMPLE 10

### Identification of A. fumigatus by PCR

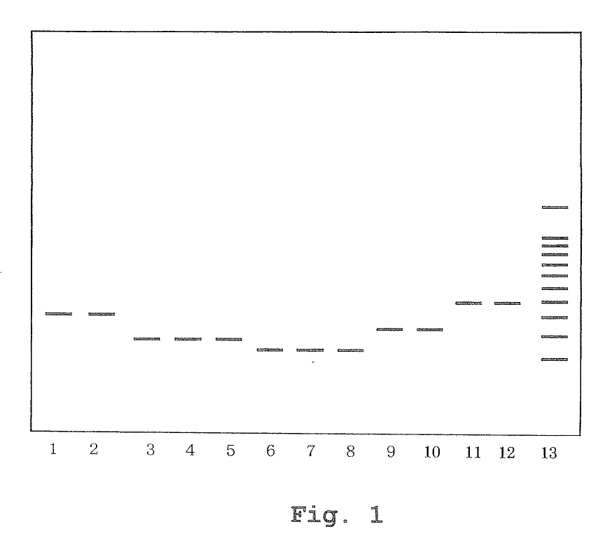

Parts of the cytochrome b genes of A. fumigatus were amplified by PCR as in Example 2 using the solutions of DNA 2 and DNA 10 (SEQ ID NO. 10) prepared in Example 1, and using TaKaRa PCR Amplification Kit (RO11) commercially available from TAKARA SHUZO CO., LTD. and a DNA amplifier (MIR-D30) commercially available from SANYO. Nucleic acids of A. fumigatus were extracted by the phenol extraction method. Ten microliters of the solution containing each of the amplified products was electrophoresed on 1% agarose gel and the gel was stained with ethidium bromide, followed by detection of fluorescence during UV irradiation (FIG. 1, lanes 1 and 2). The electrophoresis was performed at a constant voltage of 75V for 45 minutes. In addition to the reaction mixture, molecular weight markers were also electrophoresed. Based on the relative mobility, the length of the detected nucleic acid fragment was calculated to be about 323 bp.

### EXAMPLE 11

### Identification of A. flavus by PCR

By the same method as in Example 10, using solution of DNA 2, and solutions of DNA 11 (SEQ ID NO. 11) and DNA 12 (SEQ ID NO. 12) prepared in Example 1, parts of the cytochrome b genes of A. flavus were amplified by PCR and the amplified products were detected by electrophoresis (FIG. 1, lanes 3, 4 and 5). The lengths of the detected nucleic acid fragments were calculated to be about 193 bp and or about 194 bp.

### EXAMPLE 12

### Identification of A. niger by PCR

By the same method as in Example 10, using solutions of DNA 2 and DNA 13 (SEQ ID NO. 13) prepared in Example 1, parts of the cytochrome b genes of A. niger were amplified by PCR and the amplified products were detected by electrophoresis (FIG. 1, lanes 6, 7 and 8). The length of the detected nucleic acid fragments was calculated to be about 148 bp.

### EXAMPLE 13

### Identification of A. nidulans by PCR

By the same method as in Example 10, using solutions of DNA 2 and DNA 14 (SEQ ID NO. 14) prepared in Example 1, parts of the cytochrome b genes of A. nidulans were amplified by PCR and the amplified products were detected by electrophoresis (FIG. 1, lanes 9 and 10). The length of the detected nucleic acid fragments was calculated to be about 244 bp.

### EXAMPLE 14

### Identification of A. terreus by PCR

By the same method as in Example 10, using solutions of DNA 2 and DNA 15 (SEQ ID NO. 15) prepared in Example 1, parts of the cytochrome b genes of A. nidulans were amplified by PCR and the amplified products were detected by electrophoresis (FIG. 1, lanes 11 and 12). The length of the detected nucleic acid fragments was calculated to be about 397 bp.

The lanes in FIG. 1 show the results of the following strains:

Lane 1: A. fumigatus IFM40819

Lane 2: A. fumigatus IFM40806

Lane 3: A. flavus IFM41933

Lane 4: A. flavus IFM5366

Lane 5: A. flavus IFM45910

Lane 6: A. niger IFM41398

Lane 7: A. niger IFM41399

Lane 8: A. niger IFM46897

Lane 9: A. nidulans IFM47004

Lane 10: A. nidulans IFM41094

Lane 11: A. terreus IFM40850

Lane 12: A. terreus IFM41092

Lane 13: size marker (100, 200, 300, 400, 500, 600, 700, 800, 900, 1000, 1500 bp)

### EXAMPLE 15

### Amplification Reaction of Cytochrome b Gene of Cunninghamella bertholletiae

By the method as in Example 2, a part of cytochrome b gene of Cunninghamella bertholletiae IFM 46114 was amplified and sequenced. As the primers, DNA57 and DNA58 were used. As primers for sequencing, DNA57 was used as a forward primer, and DNA58 was used as a reverse primer. The determined nucleotide sequence is shown in SEQ ID NO. 76 in the SEQUENCE LISTING.

### EXAMPLE 16

### Amplification Reaction of Cytochrome b Gene of Mucor circinelloides

By the method as in Example 2, a part of cytochrome b gene of Mucor circinelloides IFM 40507 was amplified and sequenced. As the primers, DNA57, and DNA60 and DNA61 were used. As primers for sequencing, DNA57 was used as a forward primer, and DNA60 and DNA61 were used as reverse primers. The determined nucleotide sequence is shown in SEQ ID NO. 77 in the SEQUENCE LISTING.

### EXAMPLE 17

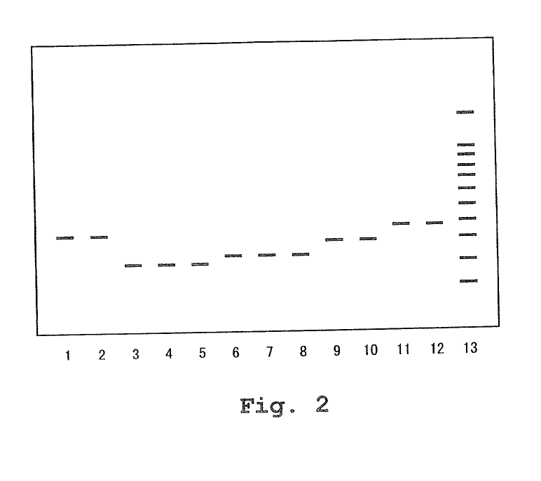

By the method as in Examples 10-14, identification of Aspergillus species was carried out. In each method, DNA57 was used as one of the primers. As other primers, DNA65 was used for identifying Aspergillus fumigatus, DNA11 and DNA12 were used for identifying Aspergillus flavus, DNA64 was used for identifying Aspergillus niger, DNA63 was used for identifying Aspergillus nidulans, and DNA62 was used for identifying Aspergillus terreus. The results of the electrophoresis are shown in FIG. 2. The strains of the respective lanes in FIG. 2 are the same as shown in FIG. 1.

### EXAMPLE 18

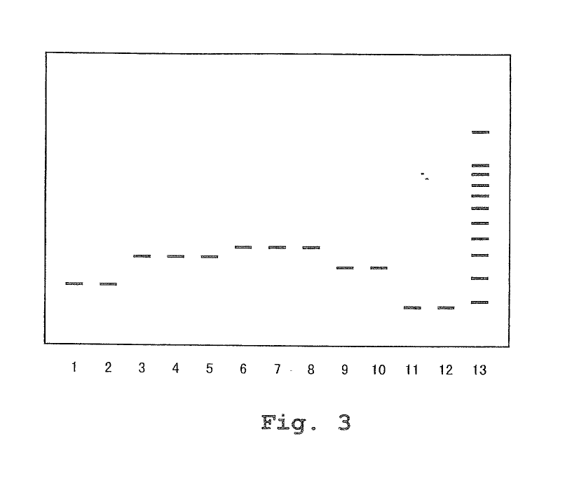

By the method as in Examples 10-14, identification of Aspergillus species was carried out. In each method, DNA3 was used as one of the primers. As other primers, DNA68 was used for identifying Aspergillus fumigatus, DNA66 and DNA67 were used for identifying Aspergillus flavus, DNA70 was used for identifying Aspergillus niger, DNA72 was used for identifying Aspergillus nidulans, and DNA74 was used for identifying Aspergillus terreus. The results of the electrophoresis are shown in FIG. 3. The strains of the respective lanes in FIG. 3 are the same as shown in FIG. 1.

### EXAMPLE 19

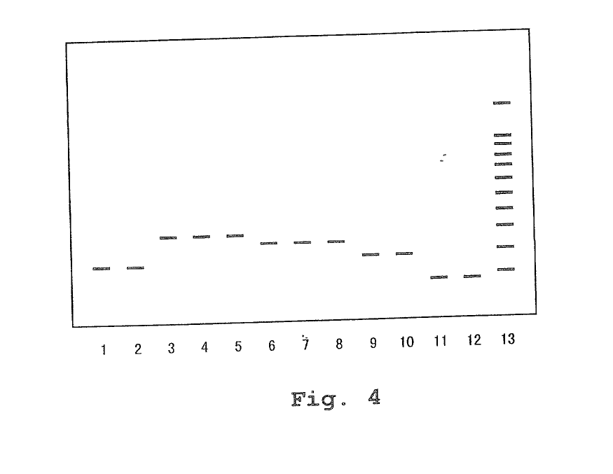

By the method as in Examples 10-14, identification of Aspergillus species was carried out. In each method, DNA58 was used as one of the primers. As other primers, DNA69 was used for identifying Aspergillus fumigatus, DNA66 and DNA67 were used for identifying Aspergillus flavus, DNA71 was used for identifying Aspergillus niger, DNA73 was used for identifying Aspergillus nidulans, and DNA75 was used for identifying Aspergillus terreus. The results of the electrophoresis are shown in FIG. 4. The strains of the respective lanes in FIG. 4 are the same as shown in FIG. 1.

### INDUSTRIAL AVAILABILITY

The present invention may be used for detection, grouping and identification of the pathogenic fungi, especially those belonging to the genus Aspergillus, quickly, simply, specifically and with high sensitivity. Therefore, the causative fungi may be rapidly identified for patients infected with pathogenic fungi, and appropriate therapy can be performed.

1. A primer for amplifying a fragment of mitochondrial cytochrome b gene of fungi, consisting essentially of a nucleic acid fragment of which nucleotide sequence is a sequence of not less than 10 consecutive nucleotides in the nucleotide sequence shown in SEQ ID NO. 57 (provided that one or more thymine at optional site(s) may be substituted with uracil) or in the nucleotide sequence complementary thereto.

2. The primer according to claim 1, which consists essentially of a nucleic acid fragment of which nucleotide sequence is shown in SEQ ID NO. 57 (provided that one or more thymine at optional site(s) may be substituted with uracil) or the nucleotide sequence complementary thereto.

3. A primer for amplifying a fragment of mitochondrial cytochrome b gene of fungi, consisting essentially of a nucleic acid fragment of which nucleotide sequence is a sequence of not less than 10 consecutive nucleotides in the nucleotide sequence shown in SEQ ID NO. 3 (provided that one or more thymine at optional site(s) may be substituted with uracil) or in the nucleotide sequence complementary thereto.

4. The primer according to claim 3, which consists essentially of a nucleic acid fragment of which nucleotide sequence is shown in SEQ ID NO. 3 (provided that one or more thymine at optional site(s) may be substituted with uracil) or the nucleotide sequence complementary thereto.

5. A primer for amplifying a fragment of mitochondrial cytochrome b gene of fungi, consisting essentially of a nucleic acid fragment of which nucleotide sequence is a sequence of not less than 10 consecutive nucleotides in the nucleotide sequence shown in SEQ ID NO. 58 (provided that one or more thymine at optional site(s) may be substituted with uracil) or in the nucleotide sequence complementary thereto.

6. The primer according to claim 5, which consists essentially of a nucleic acid fragment of which nucleotide sequence is shown in SEQ ID NO. 58 (provided that one or more thymine at optional site(s) may be substituted with uracil) or the nucleotide sequence complementary thereto.

7. A primer for amplifying a fragment of mitochondrial cytochrome b gene of fungi, consisting essentially of a nucleic acid fragment of which nucleotide sequence is a sequence of not less than 10 consecutive nucleotides in the nucleotide sequence shown in SEQ ID NO. 59 (provided that one or more thymine at optional site(s) may be substituted with uracil) or in the nucleotide sequence complementary thereto.

8. The primer according to claim 7, which consists essentially of a nucleic acid fragment of which nucleotide sequence is shown in SEQ ID NO. 59 (provided that one or more thymine at optional site(s) may be substituted with uracil) or the nucleotide sequence complementary thereto.

9. A primer for amplifying a fragment of mitochondrial cytochrome b gene of fungi, consisting essentially of a nucleic acid fragment of which nucleotide sequence is a sequence of not less than 10 consecutive nucleotides in the nucleotide sequence shown in SEQ ID NO. 60 (provided that one or more thymine at optional site(s) may be substituted with uracil) or in the nucleotide sequence complementary thereto.

10. The primer according to claim 9, which consists essentially of a nucleic acid fragment of which nucleotide sequence is shown in SEQ ID NO. 60 (provided that one or more thymine at optional site(s) may be substituted with uracil) or the nucleotide sequence complementary thereto.

11. A primer for amplifying a fragment of mitochondrial cytochrome b gene of fungi, consisting essentially of a nucleic acid fragment of which nucleotide sequence is a sequence of not less than 10 consecutive nucleotides in the nucleotide sequence shown in SEQ ID NO. 61 (provided that one or more thymine at optional site(s) may be substituted with uracil) or in the nucleotide sequence complementary thereto.

12. The primer according to claim 11, which consists essentially of a nucleic acid fragment of which nucleotide sequence is shown in SEQ ID NO. 61 (provided that one or more thymine at optional site(s) may be substituted with uracil) or the nucleotide sequence complementary thereto.

13. The primer according to any one of claims 1 to 12, wherein said primer is a mixture of said nucleic acid fragments each of which has the nucleotide sequence shown by the respective SEQ ID NO. which is a general formula.

14. The primer according to any one of claims 1 to 13, wherein said fungi are those belonging to the genus Aspergillus.

15. The primer according to any one of claims 1 to 13, wherein said fungi are those belonging to the genus Candida.

16. The primer according to any one of claims 1 to 13, wherein said fungi are those belonging to the genus Cunninghamella.

17. The primer according to any one of claims 1 to 13, wherein said fungi are those belonging to the genus Mucor.

18. The primer according to claim 14, wherein said fungi belonging to the genus Aspergillus is selected from the group consisting of Aspergillus fumigatus, Aspergillus flavus, Aspergillus niger, Aspergillus nidulans and Aspergillus terreus.

19. A nucleic acid for detection of Aspergillus fumigatus, consisting essentially of a nucleic acid fragment of which nucleotide sequence is 10 to 100 consecutive nucleotides in the nucleotide sequence shown in SEQ ID NO. 5 (provided that one or more thymine at optional site(s) may be substituted with uracil) or in the nucleotide sequence complementary thereto.

20. A nucleic acid for detection of Aspergillus flavus, consisting essentially of a nucleic acid fragment of which nucleotide sequence is a sequence of 10 to 100 consecutive nucleotides in the nucleotide sequence shown in SEQ ID NO. 6 (provided that one or more thymine at optional site(s) may be substituted with uracil) or in the nucleotide sequence complementary thereto.

21. A nucleic acid for detection of Aspergillus niger, consisting essentially of a nucleic acid fragment of which nucleotide sequence is a sequence of 10 to 100 consecutive nucleotides in the nucleotide sequence shown in SEQ ID NO. 7 (provided that one or more thymine at optional site(s) may be substituted with uracil) or in the nucleotide sequence complementary thereto.

22. A nucleic acid for detection of Aspergillus nidulans, consisting essentially of a nucleic acid fragment of which nucleotide sequence is a sequence of 10 to 100 consecutive nucleotides in the nucleotide sequence shown in SEQ ID NO. 8 (provided that one or more thymine at optional site(s) may be substituted with uracil) or in the nucleotide sequence complementary thereto.

23. A nucleic acid for detection of Aspergillus terreus, consisting essentially of a nucleic acid fragment of which nucleotide sequence is a sequence of 10 to 100 consecutive nucleotides in the nucleotide sequence shown in SEQ ID NO. 9 (provided that one or more thymine at optional site(s) may be substituted with uracil) or in the nucleotide sequence complementary thereto.

24. The nucleic acid for detection according to any one of claims 19 to 23, wherein said 10 to 100 consecutive nucleotides is 15 to 30 consecutive nucleotides.

25. A nucleic acid for detection of Aspergillus fumigatus, consisting essentially of a nucleic acid fragment of which nucleotide sequence is a sequence of not less than 10 consecutive nucleotides in the nucleotide sequence shown in SEQ ID NO. 5, the 3′-end of said nucleic acid being the 24nt “T” in the nucleotide sequence shown in SEQ ID NO. 5, or consisting essentially of a nucleic acid fragment of which nucleotide sequence is a sequence of not less than 10 consecutive nucleotides in the nucleotide sequence complementary to the sequence shown in SEQ ID NO. 5, of which 3′-end is the base that pairs with said 3′-end of said nucleic acid.

26. A nucleic acid for detection of Aspergillus fumigatus, consisting essentially of a nucleic acid fragment of which nucleotide sequence is a sequence of not less than 10 consecutive nucleotides in the nucleotide sequence shown in SEQ ID NO. 5, or consisting essentially of a nucleic acid fragment of which nucleotide sequence is a sequence of not less than 10 consecutive nucleotides in the nucleotide sequence complementary to the sequence shown in SEQ ID NO. 5, of which 3′-end is the base that pairs with said 3′-end of said nucleic acid.

27. A nucleic acid for detection of Aspergillus fumigatus, consisting essentially of a nucleic acid fragment of which nucleotide sequence is a sequence of not less than 10 consecutive nucleotides in the nucleotide sequence shown in SEQ ID NO. 5, the 3′-end of said nucleic acid being the 144nt “T” in the nucleotide sequence shown in SEQ ID NO. 5, or consisting essentially of a nucleic acid fragment of which nucleotide sequence is a sequence of not less than 10 consecutive nucleotides in the nucleotide sequence complementary to the sequence shown in SEQ ID NO. 5, of which 3′-end is the base that pairs with said 3′-end of said nucleic acid.

28. A nucleic acid for detection of Aspergillus fumigatus, consisting essentially of a nucleic acid fragment of which nucleotide sequence is a sequence of not less than 10 consecutive nucleotides in the nucleotide sequence shown in SEQ ID NO. 5, the 3′-end of said nucleic acid being the 252nt “C” in the nucleotide sequence shown in SEQ ID NO. 5, or consisting essentially of a nucleic acid fragment of which nucleotide sequence is a sequence of not less than 10 consecutive nucleotides in the nucleotide sequence complementary to the sequence shown in SEQ ID NO. 5, of which 3′-end is the base that pairs with said 3′-end of said nucleic acid.

29. A nucleic acid for detection of Aspergillus fumigatus, consisting essentially of a nucleic acid fragment of which nucleotide sequence is a sequence of not less than 10 consecutive nucleotides in the nucleotide sequence shown in SEQ ID NO. 5, the 3′-end of said nucleic acid being the 277nt “G” in the nucleotide sequence shown in SEQ ID NO. 5, or consisting essentially of a nucleic acid fragment of which nucleotide sequence is a sequence of not less than 10 consecutive nucleotides in the nucleotide sequence complementary to the sequence shown in SEQ ID NO. 5, of which 3′-end is the base that pairs with said 3′-end of said nucleic acid.

30. A nucleic acid for detection of Aspergillus fumigatus, consisting essentially of a nucleic acid fragment of which nucleotide sequence is a sequence of not less than 10 consecutive nucleotides in the nucleotide sequence shown in SEQ ID NO. 5, the 3′-end of said nucleic acid being the 304nt “G” in the nucleotide sequence shown in SEQ ID NO. 5, or consisting essentially of a nucleic acid fragment of which nucleotide sequence is a sequence of not less than 10 consecutive nucleotides in the nucleotide sequence complementary to the sequence shown in SEQ ID NO. 5, of which 3′-end is the base that pairs with said 3′-end of said nucleic acid.

31. A nucleic acid for detection of Aspergillus fumigatus, consisting essentially of a nucleic acid fragment of which nucleotide sequence is a sequence of not less than 10 consecutive nucleotides in the nucleotide sequence shown in SEQ ID NO. 5, the 3′ -end of said nucleic acid being the 312nt “A” in the nucleotide sequence shown in SEQ ID NO. 5, or consisting essentially of a nucleic acid fragment of which nucleotide sequence is a sequence of not less than 10 consecutive nucleotides in the nucleotide sequence complementary to the sequence shown in SEQ ID NO. 5, of which 3′-end is the base that pairs with said 3′-end of said nucleic acid.

32. A nucleic acid for detection of Aspergillus fumigatus, consisting essentially of a nucleic acid fragment of which nucleotide sequence is a sequence of not less than 10 consecutive nucleotides in the nucleotide sequence shown in SEQ ID NO. 5, the 3′-end of said nucleic acid being the 393nt “G” in the nucleotide sequence shown in SEQ ID NO. 5, or consisting essentially of a nucleic acid fragment of which nucleotide sequence is a sequence of not less than 10 consecutive nucleotides in the nucleotide sequence complementary to the sequence shown in SEQ ID NO. 5, of which 3′-end is the base that pairs with said 3′-end of said nucleic acid.

33. A nucleic acid for detection of Aspergillus flavus, consisting essentially of a nucleic acid fragment of which nucleotide sequence is a sequence of not less than 10 consecutive nucleotides in the nucleotide sequence shown in SEQ ID NO. 6, the 3′-end of said nucleic acid being the 174nt “Y” in the nucleotide sequence shown in SEQ ID NO. 6, or consisting essentially of a nucleic acid fragment of which nucleotide sequence is a sequence of not less than 10 consecutive nucleotides in the nucleotide sequence complementary to the sequence shown in SEQ ID NO. 6, of which 3′-end is the base that pairs with said 3′-end of said nucleic acid.

34. A nucleic acid for detection of Aspergillus niger, consisting essentially of a nucleic acid fragment of which nucleotide sequence is a sequence of not less than 10 consecutive nucleotides in the nucleotide sequence shown in SEQ ID NO. 7, the 3′-end of said nucleic acid being the 42nt “A” in the nucleotide sequence shown in SEQ ID NO. 7, or consisting essentially of a nucleic acid fragment of which nucleotide sequence is a sequence of not less than 10 consecutive nucleotides in the nucleotide sequence complementary to the sequence shown in SEQ ID NO. 7, of which 3′-end is the base that pairs with said 3′-end of said nucleic acid.

35. A nucleic acid for detection of Aspergillus niger, consisting essentially of a nucleic acid fragment of which nucleotide sequence is a sequence of not less than 10 consecutive nucleotides in the nucleotide sequence shown in SEQ ID NO. 7, the 3′-end of said nucleic acid being the 69nt “C” in the nucleotide sequence shown in SEQ ID NO. 7, or consisting essentially of a nucleic acid fragment of which nucleotide sequence is a sequence of not less than 10 consecutive nucleotides in the nucleotide sequence complementary to the sequence shown in SEQ ID NO. 7, of which 3′-end is the base that pairs with said 3′-end of said nucleic acid.

36. A nucleic acid for detection of Aspergillus niger, consisting essentially of a nucleic acid fragment of which nucleotide sequence is a sequence of not less than 10 consecutive nucleotides in the nucleotide sequence shown in SEQ ID NO. 7, the 3′-end of said nucleic acid being the 126nt “C” in the nucleotide sequence shown in SEQ ID NO. 7, or consisting essentially of a nucleic acid fragment of which nucleotide sequence is a sequence of not less than 10 consecutive nucleotides in the nucleotide sequence complementary to the sequence shown in SEQ ID NO. 7, of which 3′-end is the base that pairs with said 3′-end of said nucleic acid.

37. A nucleic acid for detection of Aspergillus niger, consisting essentially of a nucleic acid fragment of which nucleotide sequence is a sequence of not less than 10 consecutive nucleotides in the nucleotide sequence shown in SEQ ID NO. 7, the 3′-end of said nucleic acid being the 207nt “A” in the nucleotide sequence shown in SEQ ID NO. 7, or consisting essentially of a nucleic acid fragment of which nucleotide sequence is a sequence of not less than 10 consecutive nucleotides in the nucleotide sequence complementary to the sequence shown in SEQ ID NO. 7, of which 3′-end is the base that pairs with said 3′-end of said nucleic acid.

38. A nucleic acid for detection of Aspergillus niger, consisting essentially of a nucleic acid fragment of which nucleotide sequence is a sequence of not less than 10 consecutive nucleotides in the nucleotide sequence shown in SEQ ID NO. 7, the 3′-end of said nucleic acid being the 213nt “A” in the nucleotide sequence shown in SEQ ID NO. 7, or consisting essentially of a nucleic acid fragment of which nucleotide sequence is a sequence of not less than 10 consecutive nucleotides in the nucleotide sequence complementary to the sequence shown in SEQ ID NO. 7, of which 3′-end is the base that pairs with said 3′-end of said nucleic acid.

39. A nucleic acid for detection of Aspergillus niger, consisting essentially of a nucleic acid fragment of which nucleotide sequence is a sequence of not less than 10 consecutive nucleotides in the nucleotide sequence shown in SEQ ID NO. 7, the 3′-end of said nucleic acid being the 246nt “A” in the nucleotide sequence shown in SEQ ID NO. 7, or consisting essentially of a nucleic acid fragment of which nucleotide sequence is a sequence of not less than 10 consecutive nucleotides in the nucleotide sequence complementary to the sequence shown in SEQ ID NO. 7, of which 3′-end is the base that pairs with said 3′-end of said nucleic acid.

40. A nucleic acid for detection of Aspergillus niger, consisting essentially of a nucleic acid fragment of which nucleotide sequence is a sequence of not less than 10 consecutive nucleotides in the nucleotide sequence shown in SEQ ID NO. 7, the 3′-end of said nucleic acid being the 396nt “G” in the nucleotide sequence shown in SEQ ID NO. 7, or consisting essentially of a nucleic acid fragment of which nucleotide sequence is a sequence of not less than 10 consecutive nucleotides in the nucleotide sequence complementary to the sequence shown in SEQ ID NO. 7, of which 3′-end is the base that pairs with said 3′-end of said nucleic acid.

41. A nucleic acid for detection of Aspergillus nidulans, consisting essentially of a nucleic acid fragment of which nucleotide sequence is a sequence of not less than 10 consecutive nucleotides in the nucleotide sequence shown in SEQ ID NO. 8, the 3′-end of said nucleic acid being the 60nt “T” in the nucleotide sequence shown in SEQ ID NO. 8, or consisting essentially of a nucleic acid fragment of which nucleotide sequence is a sequence of not less than 10 consecutive nucleotides in the nucleotide sequence complementary to the sequence shown in SEQ ID NO. 8, of which 3′-end is the base that pairs with said 3′-end of said nucleic acid.

42. A nucleic acid for detection of Aspergillus nidulans, consisting essentially of a nucleic acid fragment of which nucleotide sequence is a sequence of not less than 10 consecutive nucleotides in the nucleotide sequence shown in SEQ ID NO. 8, the 3′-end of said nucleic acid being the 221nt “C” in the nucleotide sequence shown in SEQ ID NO. 8, or consisting essentially of a nucleic acid fragment of which nucleotide sequence is a sequence of not less than 10 consecutive nucleotides in the nucleotide sequence complementary to the sequence shown in SEQ ID NO. 8, of which 3′-end is the base that pairs with said 3′-end of said nucleic acid.

43. A nucleic acid for detection of Aspergillus nidulans, consisting essentially of a nucleic acid fragment of which nucleotide sequence is a sequence of not less than 10 consecutive nucleotides in the nucleotide sequence shown in SEQ ID NO. 8, the 3′-end of said nucleic acid being the 271nt “A” in the nucleotide sequence shown in SEQ ID NO. 8, or consisting essentially of a nucleic acid fragment of which nucleotide sequence is a sequence of not less than 10 consecutive nucleotides in the nucleotide sequence complementary to the sequence shown in SEQ ID NO. 8, of which 3′-end is the base that pairs with said 3′-end of said nucleic acid.

44. A nucleic acid for detection of Aspergillus nidulans, consisting essentially of a nucleic acid fragment of which nucleotide sequence is a sequence of not less than 10 consecutive nucleotides in the nucleotide sequence shown in SEQ ID NO. 8, the 3′-end of said nucleic acid being the 285nt “A” in the nucleotide sequence shown in SEQ ID NO. 8, or consisting essentially of a nucleic acid fragment of which nucleotide sequence is a sequence of not less than 10 consecutive nucleotides in the nucleotide sequence complementary to the sequence shown in SEQ ID NO. 8, of which 3′-end is the base that pairs with said 3′-end of said nucleic acid.

45. A nucleic acid for detection of Aspergillus nidulans, consisting essentially of a nucleic acid fragment of which nucleotide sequence is a sequence of not less than 10 consecutive nucleotides in the nucleotide sequence shown in SEQ ID NO. 8, the 3′-end of said nucleic acid being the 366nt “T” in the nucleotide sequence shown in SEQ ID NO. 8, or consisting essentially of a nucleic acid fragment of which nucleotide sequence is a sequence of not less than 10 consecutive nucleotides in the nucleotide sequence complementary to the sequence shown in SEQ ID NO. 8, of which 3′-end is the base that pairs with said 3′-end of said nucleic acid.

46. A nucleic acid for detection of Aspergillus nidulans, consisting essentially of a nucleic acid fragment of which nucleotide sequence is a sequence of not less than 10 consecutive nucleotides in the nucleotide sequence shown in SEQ ID NO. 8, the 3′-end of said nucleic acid being the 387nt “A” in the nucleotide sequence shown in SEQ ID NO. 8, or consisting essentially of a nucleic acid fragment of which nucleotide sequence is a sequence of not less than 10 consecutive nucleotides in the nucleotide sequence complementary to the sequence shown in SEQ ID NO. 8, of which 3′-end is the base that pairs with said 3′-end of said nucleic acid.

47. A nucleic acid for detection of Aspergillus terreus, consisting essentially of a nucleic acid fragment of which nucleotide sequence is a sequence of not less than 10 consecutive nucleotides in the nucleotide sequence shown in SEQ ID NO. 9, the 3′-end of said nucleic acid being the 25nt “T” in the nucleotide sequence shown in SEQ ID NO. 9, or consisting essentially of a nucleic acid fragment of which nucleotide sequence is a sequence of not less than 10 consecutive nucleotides in the nucleotide sequence complementary to the sequence shown in SEQ ID NO. 9, of which 3′-end is the base that pairs with said 3′-end of said nucleic acid.

48. A nucleic acid for detection of Aspergillus terreus, consisting essentially of a nucleic acid fragment of which nucleotide sequence is a sequence of not less than 10 consecutive nucleotides in the nucleotide sequence shown in SEQ ID NO. 9, the 3′-end of said nucleic acid being the 48nt “T” in the nucleotide sequence shown in SEQ ID NO. 9, or consisting essentially of a nucleic acid fragment of which nucleotide sequence is a sequence of not less than 10 consecutive nucleotides in the nucleotide sequence complementary to the sequence shown in SEQ ID NO. 9, of which 3′-end is the base that pairs with said 3′-end of said nucleic acid.

49. A nucleic acid for detection of Aspergillus terreus, consisting essentially of a nucleic acid fragment of which nucleotide sequence is a sequence of not less than 10 consecutive nucleotides in the nucleotide sequence shown in SEQ ID NO. 9, the 3′-end of said nucleic acid being the 162nt “T” in the nucleotide sequence shown in SEQ ID NO. 9, or consisting essentially of a nucleic acid fragment of which nucleotide sequence is a sequence of not less than 10 consecutive nucleotides in the nucleotide sequence complementary to the sequence shown in SEQ ID NO. 9, of which 3′-end is the base that pairs with said 3′-end of said nucleic acid.

50. A nucleic acid for detection of Aspergillus terreus, consisting essentially of a nucleic acid fragment of which nucleotide sequence is a sequence of not less than 10 consecutive nucleotides in the nucleotide sequence shown in SEQ ID NO. 9, the 3′-end of said nucleic acid being the 225nt “C” in the nucleotide sequence shown in SEQ ID NO. 9, or consisting essentially of a nucleic acid fragment of which nucleotide sequence is a sequence of not less than 10 consecutive nucleotides in the nucleotide sequence complementary to the sequence shown in SEQ ID NO. 9, of which 3′-end is the base that pairs with said 3′-end of said nucleic acid.

51. A nucleic acid for detection of Aspergillus terreus, consisting essentially of a nucleic acid fragment of which nucleotide sequence is a sequence of not less than 10 consecutive nucleotides in the nucleotide sequence shown in SEQ ID NO. 9, the 3′-end of said nucleic acid being the 261nt “C” in the nucleotide sequence shown in SEQ ID NO. 9, or consisting essentially of a nucleic acid fragment of which nucleotide sequence is a sequence of not less than 10 consecutive nucleotides in the nucleotide sequence complementary to the sequence shown in SEQ ID NO. 9, of which 3′-end is the base that pairs with said 3′-end of said nucleic acid.

52. A nucleic acid for detection of Aspergillus terreus, consisting essentially of a nucleic acid fragment of which nucleotide sequence is a sequence of not less than 10 consecutive nucleotides in the nucleotide sequence shown in SEQ ID NO. 9, the 3′-end of said nucleic acid being the 339nt “C” in the nucleotide sequence shown in SEQ ID NO. 9, or consisting essentially of a nucleic acid fragment of which nucleotide sequence is a sequence of not less than 10 consecutive nucleotides in the nucleotide sequence complementary to the sequence shown in SEQ ID NO. 10, of which 3′-end is the base that pairs with said 3′-end of said nucleic acid.

53. A nucleic acid for detection of Aspergillus terreus, consisting essentially of a nucleic acid fragment of which nucleotide sequence is a sequence of not less than 10 consecutive nucleotides in the nucleotide sequence shown in SEQ ID NO. 9, the 3′-end of said nucleic acid being the 360nt “A” in the nucleotide sequence shown in SEQ ID NO. 9, or consisting essentially of a nucleic acid fragment of which nucleotide sequence is a sequence of not less than 10 consecutive nucleotides in the nucleotide sequence complementary to the sequence shown in SEQ ID NO. 9, of which 3′-end is the base that pairs with said 3′-end of said nucleic acid.

54. A nucleic acid for detection of Aspergillus terreus, consisting essentially of a nucleic acid fragment of which nucleotide sequence is a sequence of not less than 10 consecutive nucleotides in the nucleotide sequence shown in SEQ ID NO. 9, the 3′-end of said nucleic acid being the 375nt “A” in the nucleotide sequence shown in SEQ ID NO. 9, or consisting essentially of a nucleic acid fragment of which nucleotide sequence is a sequence of not less than 10 consecutive nucleotides in the nucleotide sequence—complementary to the sequence shown in SEQ ID NO. 9, of which 3′-end is the base that pairs with said 3′-end of said nucleic acid.

55. A nucleic acid for detection of Aspergillus terreus, consisting essentially of a nucleic acid fragment of which nucleotide sequence is a sequence of not less than 10 consecutive nucleotides in the nucleotide sequence shown in SEQ ID NO. 9, the 3′-end of said nucleic acid being the 411nt “A” in the nucleotide sequence shown in SEQ ID NO. 9, or consisting essentially of a nucleic acid fragment of which nucleotide sequence is a sequence of not less than 10 consecutive nucleotides in the nucleotide sequence complementary to the sequence shown in SEQ ID NO. 9, of which 3′-end is the base that pairs with said 3′-end of said nucleic acid.

56. The nucleic acid according to any one of claims 25 to 55, wherein the number of nucleotides therein is 10 to 100.

57. The nucleic acid according to claim 56, wherein the number of nucleotides therein is 15 to 30.

58. The nucleic acid according to any one of claims 25 to 57, wherein said nucleic acid is a primer for amplification of a nucleic acid.

59. The nucleic acid according to any one of claims 25 to 57, wherein said nucleic acid is a labeled probe.

60. The nucleic acid for detection of Aspergillus fumigatus according to claim 30, wherein said nucleic acid consists essentially of a nucleic acid fragment of which nucleotide sequence is the sequence of shown in SEQ ID NO. 10 (provided that one or more thymine at optional site(s) may be substituted with uracil), or consists essentially of a nucleic acid fragment complementary to said nucleic acid.

61. The nucleic acid for detection of Aspergillus flavus according to claim 33, wherein said nucleic acid consists essentially of a nucleic acid fragment of which nucleotide sequence is the sequence of shown in SEQ ID NO. 11 (provided that one or more thymine at optional site(s) may be substituted with uracil), or consists essentially of a nucleic acid fragment complementary to said nucleic acid.

62. The nucleic acid for detection of Aspergillus flavus according to claim 33, wherein said nucleic acid consists essentially of a nucleic acid fragment of which nucleotide sequence is the sequence of shown in SEQ ID NO. 12 (provided that one or more thymine at optional site(s) may be substituted with uracil), or consists essentially of a nucleic acid fragment complementary to said nucleic acid.

63. The nucleic acid for detection of Aspergillus niger according to claim 36, wherein said nucleic acid consists essentially of a nucleic acid fragment of which nucleotide sequence is the sequence of shown in SEQ ID NO. 13 (provided that one or more thymine at optional site(s) may be substituted with uracil), or consists essentially of a nucleic acid fragment complementary to said nucleic acid.

64. The nucleic acid for detection of Aspergillus nidulans according to claim 42, wherein said nucleic acid consists essentially of a nucleic acid fragment of which nucleotide sequence is the sequence of shown in SEQ ID NO. 14 (provided that one or more thymine at optional site(s) may be substituted with uracil), or consists essentially of a nucleic acid fragment complementary to said nucleic acid.

65. The nucleic acid for detection of Aspergillus terreus according to claim 54, wherein said nucleic acid consists essentially of a nucleic acid fragment of which nucleotide sequence is the sequence of shown in SEQ ID NO. 15 (provided that one or more thymine at optional site(s) may be substituted with uracil), or consists essentially of a nucleic acid fragment complementary to said nucleic acid.

66. The nucleic acid for detection of Aspergillus terreus according to claim 53, wherein said nucleic acid consists essentially of a nucleic acid fragment of which nucleotide sequence is the sequence of shown in SEQ ID NO. 62 (provided that one or more thymine at optional site(s) may be substituted with uracil), or consists essentially of a nucleic acid fragment complementary to said nucleic acid.

67. The nucleic acid for detection of Aspergillus nidulans according to claim 43, wherein said nucleic acid consists essentially of a nucleic acid fragment of which nucleotide sequence is the sequence of shown in SEQ ID NO. 63 (provided that one or more thymine at optional site(s) may be substituted with uracil), or consists essentially of a nucleic acid fragment complementary to said nucleic acid.

68. The nucleic acid for detection of Aspergillus niger according to claim 37, wherein said nucleic acid consists essentially of a nucleic acid fragment of which nucleotide sequence is the sequence of shown in SEQ ID NO. 64 (provided that one or more thymine at optional site(s) may be substituted with uracil), or consists essentially of a nucleic acid fragment complementary to said nucleic acid.

69. The nucleic acid for detection of Aspergillus fumigatus according to claim 30, wherein said nucleic acid consists essentially of a nucleic acid fragment of which nucleotide sequence is the sequence of shown in SEQ ID NO. 65 (provided that one or more thymine at optional site(s) may be substituted with uracil), or consists essentially of a nucleic acid fragment complementary to said nucleic acid.

70. The nucleic acid for detection of Aspergillus flavus according to claim 37, wherein said nucleic acid consists essentially of a nucleic acid fragment of which nucleotide sequence is the sequence of shown in SEQ ID NO. 66 (provided that one or more thymine at optional site(s) may be substituted with uracil), or consists essentially of a nucleic acid fragment complementary to said nucleic acid.

71. The nucleic acid for detection of Aspergillus flavus according to claim 37, wherein said nucleic acid consists essentially of a nucleic acid fragment of which nucleotide sequence is the sequence of shown in SEQ ID NO. 67 (provided that one or more thymine at optional site(s) may be substituted with uracil), or consists essentially of a nucleic acid fragment complementary to said nucleic acid.

72. The nucleic acid for detection of Aspergillus fumigatus according to claim 30, wherein said nucleic acid consists essentially of a nucleic acid fragment of which nucleotide sequence is the sequence of shown in SEQ ID NO. 68 (provided that one or more thymine at optional site(s) may be substituted with uracil), or consists essentially of a nucleic acid fragment complementary to said nucleic acid.

73. The nucleic acid for detection of Aspergillus fumigatus according to claim 31, wherein said nucleic acid consists essentially of a nucleic acid fragment of which nucleotide sequence is the sequence of shown in SEQ ID NO. 69 (provided that one or more thymine at optional site(s) may be substituted with uracil), or consists essentially of a nucleic acid fragment complementary to said nucleic acid.

74. The nucleic acid for detection of Aspergillus niger according to claim 36, wherein said nucleic acid consists essentially of a nucleic acid fragment of which nucleotide sequence is the sequence of shown in SEQ ID NO. 70 (provided that one or more thymine at optional site(s) may be substituted with uracil), or consists essentially of a nucleic acid fragment complementary to said nucleic acid.

75. The nucleic acid for detection of Aspergillus niger according to claim 38, wherein said nucleic acid consists essentially of a nucleic acid fragment of which nucleotide sequence is the sequence of shown in SEQ ID NO. 71 (provided that one or more thymine at optional site(s) may be substituted with uracil), or consists essentially of a nucleic acid fragment complementary to said nucleic acid.

76. The nucleic acid for detection of Aspergillus nidulans according to claim 42, wherein said nucleic acid consists essentially of a nucleic acid fragment of which nucleotide sequence is the sequence of shown in SEQ ID NO. 72 (provided that one or more thymine at optional site(s) may be substituted with uracil), or consists essentially of a nucleic acid fragment complementary to said nucleic acid.

77. The nucleic acid for detection of Aspergillus nidulans according to claim 44, wherein said nucleic acid consists essentially of a nucleic acid fragment of which nucleotide sequence is the sequence of shown in SEQ ID NO. 73 (provided that one or more thymine at optional site(s) may be substituted with uracil), or consists essentially of a nucleic acid fragment complementary to said nucleic acid.

78. The nucleic acid for detection of Aspergillus terreus according to claim 54, wherein said nucleic acid consists essentially of a nucleic acid fragment of which nucleotide sequence is the sequence of shown in SEQ ID NO. 74 (provided that one or more thymine at optional site(s) may be substituted with uracil), or consists essentially of a nucleic acid fragment complementary to said nucleic acid.

79. The nucleic acid for detection of Aspergillus terreus according to claim 54, wherein said nucleic acid consists essentially of a nucleic acid fragment of which nucleotide sequence is the sequence of shown in SEQ ID NO. 75 (provided that one or more thymine at optional site(s) may be substituted with uracil), or consists essentially of a nucleic acid fragment complementary to said nucleic acid.

80. The primer according to claim 1 or 2, which is a forward primer.

81. The nucleic acid according to any one of claims 70 to 79, which is a forward primer.

82. The primer according to any one of claims 3 to 11, which is a reverse primer.

83. The nucleic acid according to any one of claims 60 to 69, which is a reverse primer.

84. A method for detecting and/or identifying fungi belonging to the genus Aspergillus comprising amplifying a fragment of mitochondrial cytochrome b gene of fungi belonging to the genus Aspergillus by a gene amplification method using at least one forward primer selected from the group consisting of nucleic acid fragments of which nucleotide sequence is a sequence of not less than 10 consecutive nucleotides in the nucleotide sequence shown in SEQ ID NO. 1 or 2 (provided that one or more thymine at optional site(s) may be substituted with uracil) and the forward primer according to claim 80, and at least one reverse primer according to claim 83.

85. A method for detecting and/or identifying fungi belonging to the genus Aspergillus comprising amplifying a fragment of mitochondrial cytochrome b gene of fungi belonging to the genus Aspergillus by a gene amplification method using at least one forward primer according to claim 81, and at least one reverse primer selected from the group consisting of the reverse primer according to claim 82 and nucleic acid fragments of which nucleotide sequence is a sequence of not less than 10 consecutive nucleotides in the nucleotide sequence shown in SEQ ID NO. 4 (provided that one or more thymine at optional site(s) may be substituted with uracil).

86. A method for detecting and/or identifying fungi belonging to the genus Aspergillus comprising amplifying a fragment of mitochondrial cytochrome b gene of fungi belonging to the genus Aspergillus by a gene amplification method using at least one forward primer selected from the group consisting of nucleic acid fragments of which nucleotide sequence is a sequence of not less than 10 consecutive nucleotides in the nucleotide sequence shown in SEQ ID NO. 1 or 2 (provided that one or more thymine at optional site(s) may be substituted with uracil) and the forward primer according to claim 80, and at least one reverse primer selected from the group consisting of the reverse primer according to claim 82 and nucleic acid fragments of which nucleotide sequence is a sequence of not less than 10 consecutive nucleotides in the nucleotide sequence shown in SEQ ID NO. 4 (provided that one or more thymine at optional site(s) may be substituted with uracil).

87. A DNA fragment having a nucleotide sequence shown in any one of SEQ ID NOs. 5-9.
